(19) United States
(12) Patent Application Publication
(10) Pub. No.: US 2023/0175955 A1
(43) Pub. Date: Jun. 8, 2023

(54) EVALUATION OF BRAIN TISSUE AND MATERIAL BASED ON A FRACTION-PRODUCT AND OPTICAL SPECTROSCOPY

(71) Applicant: United States Government as Represented by the Department of Veterans Affairs, Washington, DC (US)

(72) Inventors: Frank A. Greco, Washington, DC (US); Eugene B. Hanlon, Washington, DC (US)

(21) Appl. No.: 18/074,364

(22) Filed: Dec. 2, 2022

Related U.S. Application Data
(60) Provisional application No. 63/285,389, filed on Dec. 2, 2021.

(51) Int. Cl. G01N 21/31

(52) U.S. Cl. CPC G01N 21/31; A61B 5/4088

(57) ABSTRACT

The methods, apparatuses, computer-readable media, and systems described enable regions of electromagnetic spectra that may distinguish different biological specimens to be determined. Regions of electromagnetic spectra that distinguish known biological specimens then become candidates for methods to classify unknown specimens and/or make a medical diagnosis. A fraction-product, determined from two arrays associated with two groups, may be used to determine optimal discriminants for the two groups given numerical measurements of particular properties of the members of both groups.

### CROSS-REFERENCE TO RELATED APPLICATION

This application claims the benefit of and priority to U.S. Provisional Patent Application No. 63/285,389, filed Dec. 2, 2021, the contents of which application are hereby incorporated by reference herein in their entireties.

### BACKGROUND

Spectra of electromagnetic radiation may be used to determine and/or define a chemical composition of a material of interest. However, the material of biological tissues such as brain/neurological tissue is complex and that complexity is reflected in the spectra. Although two different biological specimens (e.g., a specimen with Alzheimer's disease vs. a normal (age-matched) specimen, a specimen with Lewy bodies vs. a specimen without Lewy bodies, a specimen affected by Gulf War Illness (GWI) vs. specimens unaffected by GWI, etc.) will give rise to different spectra, discovering the regions of difference may be daunting. Modern spectrometers may record thousands of spectral intensities, yet the human eye is unable to discern differences between two spectra from specimens that are known to be distinct. As such, determining/discovering regions of electromagnetic spectra that may distinguish different biological specimens, such as specimens affected by one or more brain ailments, injuries, diseases, and/or the like is difficult.

### SUMMARY

It is to be understood that both the following general description and the following detailed description are exemplary and explanatory only and are not restrictive. Methods, apparatuses, computer-readable media, and systems for evaluating brain tissue and material based on a fraction-product and/or optical spectroscopy are described. Regions of electromagnetic spectra may be determined and used to distinguish different biological specimens (e.g., a specimen with Alzheimer's disease vs. a normal (age-matched) specimen, a specimen with Lewy bodies vs. a specimen without Lewy bodies, a specimen affected by Gulf War Illness (GWI) vs. specimens unaffected by GWI, etc.). Regions of spectra that distinguish known biological specimens then become candidates for methods to classify unknown specimens, that is, to make a medical diagnosis. In order to apply the embodiments of this disclosure (e,g., the methods of this disclosure) to actual data, the data can be divided into two non-empty subsets. In a first subset, the fraction-product can be applied to discover features that best separate disease from normal. In models using adaptive parameters, this first subset is usually called the “training set” because the parameters may be adjusted for optimal performance. In this disclosure, when implemented, the fraction-product has avoided the need to adjust parameters. As such, rather than referring to a training set, this disclosure refers to that first subset as a “discovery set” for two reasons: (1) it fits better with the notion of exploratory data analysis; and (2) in cases that do not require weighting of features, referring to a discovery set avoids the connotation of adjusting parameters that the term “training” carries. After satisfactory features (e.g., best features, second best features, a combination thereof, etc.) have been discovered, these are then applied to a second subset of the two non-empty subsets of data, referred to as the “test set,” for example, in order to confirm the efficacy of these discovered features to separate disease from normal.

Described are methods comprising, receiving, by a computing device, a first array of numbers and a second array of numbers, wherein the first array and the second array are associated with a common index set, wherein the common index set is associated with a plurality of index elements; determining: (a) at each index element of the plurality of index elements, for the first array and the second array, a median of numeric values of members of the respective array, (b) at each index element of the plurality of index elements, based on an average of the two median values determined at (a), a taxonomic cut-off value, (c) for the respective array associated with the greater median value between the two median values, a fraction of the members of the respective array whose numeric values exceed the taxonomic cut-off value determined at (b), and (d) for the respective array associated with the lesser median value between the two median values, the fraction of the members of the respective array whose numeric values are less than the taxonomic cut-off value determined at (b); determining, at each index element of the plurality of index elements, a respective fraction-product value, wherein the respective fraction-product value is based on a product of the respective value determined at step (c) and the respective value determined at step (d); determining, based on each of the respective fraction-product values, a third array of numbers associated with the common index set; and determining, based on the third array, one or more optimal discriminants for separating a first group and a second group, wherein the first group is associated with the first array and the second group is associated with the second array, wherein the one or more optimal discriminants are equal to the largest value of the third array.

Determining the one or more optimal discriminants comprises: (1) selecting the largest fraction-product value of the fraction-product values associated with the third array, (2) selecting index elements of the third array associated with the largest fraction-product value, (3) assessing, based on the index elements of the third array selected at step (2), a separation of the first group and the second group, (4) if the separation is inadequate, determining a fraction-product value less than the largest fraction-product value, (5) selecting index elements of the third array associated with the fraction-product value less than the largest fraction-product value, (6) assessing, based on the index elements of the third array selected at step (5), a separation of the first group and the second group, and (7) if the separation is inadequate, repeating steps (4)-(7). The index elements selected by the fraction-product are candidate discriminant statistics. With respect to a separation being adequate, in many cases, complete separation without regard to magnitude of separation can be considered adequate. Otherwise, adequate separation might be judged with respect to clinical relevance. For example, if Alzheimer's disease (AD) is correctly diagnosed clinically 65% of the time, then separation equal to or greater than 0.7 might be considered adequate.

Throughout steps (4)-(7), the correlation coefficients obtained by pairwise comparison between distinct index elements within the first array and the second array of these candidate discriminants may be used to prioritize those which are uncorrelated. In this disclosure, the term “statistic” is used in the mathematical sense of a function whose domain is a random variable (with the term “variate” being a synonym for random variable).

Also described are computer-implemented methods comprising: receiving first optical spectra and second optical spectra; determining, based on an average of median values of spectral intensity for wavelengths present in each of the first optical spectra and each of the second optical spectra, a diagnostic cut-off value; determining, based on the diagnostic cut-off value, a first quantity of the first optical spectra and a second quantity of the second optical spectra; determining, based on the first quantity and the second quantity, a threshold value; and classifying, based on the threshold value, a subset of the first optical spectra and the second optical spectra as candidate optical features. The candidate optical features form an array that, in this disclosure is oftentimes referred to as “third array.” Each candidate optical feature comprises one wavelength having a spectral value—that is, a value at that wavelength—that meets or exceeds the threshold value. That spectral value can be a fraction-product. It is noted that there are two kinds of candidate optical features: one at each pixel with an fₚ-value that exceeds the level for significance (which may be due to chance) and one for which the fₚ-value is significant on contiguous pixels and is therefore more likely to correspond to a real optical feature with linewidth.

Also described are computer-implemented methods comprising: (1) collecting spectral data associated with two groups of specimens that are known to differ in a significant manner (disease, non-disease). For example, the two groups may be labeled group A and group B; (2) For each wavelength measured from a spectrum determined from each of the two groups of specimens/subjects: (a) determining a median value of spectral intensity for members of the group A; (b) determining a median value of spectral intensity for members of the group B; (c) assigning a diagnostic cut-off value for distinguishing the group A and the group B as an average of the two median values; (d) determining a fraction f_{A} of the members of the group A that are correctly classified by the diagnostic cut-off value; (e) determining a fraction f_{B}of the members of the group B that are correctly classified by the diagnostic cut-off value; (0 determining the product of the fractions f_{A} and f_{B} determined at step (d) and step (e) as a coarse measure of efficiency. Values determined at step (0 are positive and range from 0.25 (randomness) to 1.0 (complete separation); and (3) determining candidate wavelengths for further study to classify unknowns as A or B based on values calculated at step (f). Further study can include paring down the number of candidate features (e.g., optical discriminants) that are correlated beyond a threshold correlation coefficient. Additionally, linear discriminant analysis (LDA) and/or principal component analysis (PCA) can then be applied, using a pared down group of candidate features. Further, or in some cases, further study can include generating scatter plots for exploratory data analysis.

This summary is not intended to identify critical or essential features of the disclosure, but merely to summarize certain features and variations thereof. Other details and features will be described in the sections that follow.

### BRIEF DESCRIPTION OF THE DRAWINGS

The accompanying drawings, which are incorporated in and constitute a part of this specification, together with the description, serve to explain the principles of the methods and systems:

FIG. 1 shows an example flowchart for analyzing and processing optical spectra data used to evaluate brain tissue and material based on a fraction-product;

FIG. 2 a block diagram of an example system for implementing evaluation of brain tissue and material based on a fraction-product and optical spectroscopy;

FIG. 3 shows result from simulations for evaluating brain tissue and material based on a fraction-product;

FIG. 4 shows a summary of statistics for evaluating brain tissue and material based on a fraction-product;

FIG. 5 shows results of principal component analysis and linear discriminant analysis;

FIG. 6 shows the similarity of optical spectra from biological tissue and serves to underscore how the fraction-product may be useful in finding spectral features that are useful for classification;

FIG. 7 shows the general behavior of the factors that affect the value of a fraction-product;

FIG. 8 shows the effect of varying a number of pixels in the camera of the spectrometer and the number of subjects in each group on the distribution of a fraction-product;

FIG. 9 shows how the number of subjects in each group affects the value of the fraction-product above which no values should occur by chance as determined by simulations;

FIGS. 10A-10D shows the effect small numbers of subjects have on the range of values that the fraction-product may assume and on the distribution, where FIG. 10A shows results for five subjects, FIG. 10B shows results for seven subjects,

FIG. 10C shows results for 10 subjects, and FIG. 10D shows results for 30 subjects;

FIG. 11 shows how choosing diagnostic cut-offs values of 0.8, 0.7 and 0.6 for a fraction-product includes increasingly greater regions of an optical spectrum;

FIG. 12 shows a summary of demographic information;

FIG. 13 shows detailed pathologic information on subjects with Lewy bodies;

FIG. 14 shows a schematic of a plastic template probe to illustrate how optical spectra can be acquired at a subject's temple;

FIG. 15 shows a summary of empiric findings relating to synucleinopathy to support the hypothesis that transneuronal degeneration explains our ability to detect Lewy bodies outside the temporal lobe by optical properties of the temporal lobe;

FIG. 16 shows utility of fraction-product by plotting two subjects with Lewy bodies in the temporal lobe, comparing their classification to those subjects with Lewy bodies outside of the temporal lobe, and demonstrating that all are properly classified;

FIG. 17 shows the utility of fraction-product by the complete separation of subjects with Alzheimer's disease without other significant pathology from controls by plotting unweighted classifiers identified by embodiments of this disclosure;

FIG. 18 demonstrates that plotting subjects with Alzheimer's disease and additional pathology using the same unweighted classifier in FIG. 17 still separates all Alzheimer's subjects from controls;

FIG. 19 shows that the same classifiers used in FIG. 17 and FIG. 18 also separates subjects with more and less severe forms of mild cognitive impairment;

FIG. 20 shows principal component analysis of the subjects plotted in FIG. 17 and FIG. 19 and how the scores assigned to the mildly cognitively impaired subjects objectively divides them into two groups;

FIG. 21 shows a flowchart of an example method for evaluating brain tissue and material based on a fraction-product;

FIG. 22 shows a flowchart of an example method for evaluating brain tissue and material based on a fraction-product; and

FIG. 23 shows a flowchart of an example method for evaluating optical spectra related to brain tissue and material based on a fraction-product.

### DETAILED DESCRIPTION

As used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another configuration includes from the one /particular value and/or to the other particular value. When values are expressed as approximations, by use of the antecedent “about,” it will be understood that the particular value forms another configuration. It will be further understood that the endpoints of each of the ranges are significant both in relation to the other endpoint, and independently of the other endpoint.

“Optional” or “optionally” means that the subsequently described event or circumstance may or may not occur, and that the description includes cases where said event or circumstance occurs and cases where it does not.

Throughout the description and claims of this specification, the word “comprise” and variations of the word, such as “comprising” and “comprises,” means “including but not limited to,” and is not intended to exclude other components, integers or steps. “Exemplary” means “an example of” and is not intended to convey an indication of a preferred or ideal configuration. “Such as” is not used in a restrictive sense, but for explanatory purposes. Further, the words “diagnostic,” “classification,” and “taxonomic” are synonyms of one another and, thus, are used interchangeably.

It is understood that when combinations, subsets, interactions, groups, etc. of components are described that, while specific reference of each various individual and collective combinations and permutations of these may not be explicitly described, each is specifically contemplated and described herein. This applies to all parts of this application including, but not limited to, steps in described methods. Thus, if there are a variety of additional steps that may be performed it is understood that each of these additional steps may be performed with any specific configuration or combination of configurations of the described methods.

As will be appreciated by one skilled in the art, hardware, software, or a combination of software and hardware may be implemented. Furthermore, a computer program product on a computer-readable storage medium (e.g., non-transitory) having processor-executable instructions (e.g., computer software) embodied in the storage medium. Any suitable computer-readable storage medium may be utilized including hard disks, CD-ROMs, optical storage devices, magnetic storage devices, memresistors, Non-Volatile Random Access Memory (NVRAM), flash memory, or a combination thereof

Throughout this application reference is made to block diagrams and flowcharts. It will be understood that each block of the block diagrams and flowcharts, and combinations of blocks in the block diagrams and flowcharts, respectively, may be implemented by processor-executable instructions. These processor-executable instructions may be loaded onto a general purpose computer, special purpose computer, or other programmable data processing apparatus to produce a machine, such that the processor-executable instructions which execute on the computer or other programmable data processing apparatus create a device for implementing the functions specified in the flowchart block or blocks.

These processor-executable instructions may also be stored in a computer-readable memory that may direct a computer or other programmable data processing apparatus to function in a particular manner, such that the processor-executable instructions stored in the computer-readable memory produce an article of manufacture including processor-executable instructions for implementing the function specified in the flowchart block or blocks. The processor-executable instructions may also be loaded onto a computer or other programmable data processing apparatus to cause a series of operational steps to be performed on the computer or other programmable apparatus to produce a computer-implemented process such that the processor-executable instructions that execute on the computer or other programmable apparatus provide steps for implementing the functions specified in the flowchart block or blocks.

Accordingly, blocks of the block diagrams and flowcharts support combinations of devices for performing the specified functions, combinations of steps for performing the specified functions and program instruction means for performing the specified functions. It will also be understood that each block of the block diagrams and flowcharts, and combinations of blocks in the block diagrams and flowcharts, may be implemented by special purpose hardware-based computer systems that perform the specified functions or steps, or combinations of special purpose hardware and computer instructions.

This detailed description may refer to a given entity performing some action. It should be understood that this language may in some cases mean that a system (e.g., a computer) owned and/or controlled by the given entity is actually performing the action.

Methods, apparatuses, computer-readable media, and systems for evaluating brain tissue and material based on a fraction-product are described. Regions of electromagnetic spectra may be determined and used to distinguish different biological specimens (e.g., a specimen with Alzheimer's disease vs. a normal (age-matched) specimen, a specimen with Lewy bodies vs. a specimen without Lewy bodies, a specimen affected by Gulf War Illness (GWI) vs. specimens unaffected by GWI, etc.). Regions of electromagnetic spectra that distinguish known biological specimens then become candidate discriminants for methods to classify unknown specimens, that is, to make a medical diagnosis. The methods described herein may be used to evaluate and process data (e.g., spectral data) to analyze brain tissue and material based on a fraction-product, such as brain tissue and material associated with different biological specimens (e.g., a specimen with Alzheimer's disease vs. a normal (age-matched) specimen, a specimen with Lewy bodies vs. a specimen without Lewy bodies, a specimen affected by Gulf War Illness (GWI) vs. specimens unaffected by GWI, etc.).

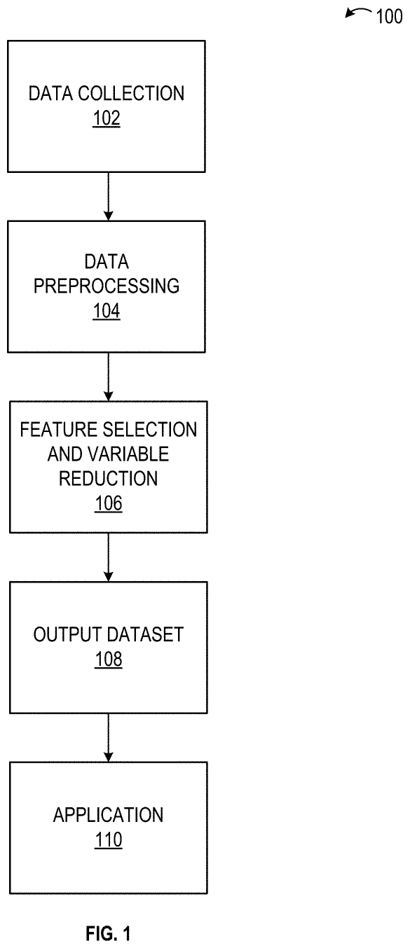

FIG. 1 is an example flowchart 100 for data/information analysis and processing used to evaluate brain tissue and material based on a fraction-product and, in some cases, optical spectroscopy. A fraction-product may be used for the “discovery” of features, for example, associated with optical spectra, that may be useful for discriminating between two groups. Empirically, the fraction-product may be used for improved analysis of small sample sizes in comparison to, for example, Mahalanobis distance. A determined fraction-product, as described herein, may be used for/with principal component analysis (PCA) for dimensionality reduction for example. The fraction-product may be used to determine/identify classifiers for any numerical data set for which the classification scheme is binary. The fraction-product may be used to determine differences between the two groups, for example, spectral regions where the two groups may differ, when the difference between the two groups is not apparent by inspection.

At block 102, data records (and/or datasets) may first be collected to determine relevant variables. For example, data records for two groups (group A and group B) of specimens that are known to differ in a significant manner (e.g., disease vs. non-disease, Alzheimer's disease vs normal, Lewy bodies in brain tissue vs. no Lewy bodies in brain tissue, Gulf War illness identified in blood samples vs. no Gulf War illness identified in blood samples, etc.). Data records may be collected by any appropriate technique. For example, data records may be collected from specimens, services, an application, an entity, and/or the like.

At block 104, the data records may be pre-processed to remove obvious erroneous or inconsistent data records. Such pre-processing may be referred to as data normalization.

At block 106, pre-processed data may be provided to an algorithm, such as a fraction-product algorithm. The fraction-product algorithm enables the determination/identification of the features, such as features associated with optical spectra that may be useful for discriminating between two groups, and the adjustment of parameters of any model used afterward. For example, the fraction-product algorithm may be used to reduce any number of potential parameters to a desired subset of parameters. The reduced subset of variables may be used to create accurate data models.

At block 108, the reduced subset of variables may further be outputted to a data storage for later retrieval.

At block 110, the reduced subset of variables may be outputted to other application software programs to further analyze and/or model the data set. For example, three-dimensional (3D) and two-dimensional (2D) plots of spectral features may be determined, previously undiscoverable distinctions between two groups may be determined, and spectral aspects of brain tissue and material may be evaluated. The fraction-product described herein provides a statistical argument that reduces the need for “discovery” and “test” sets within volumes of data. For example, for optical spectra, different optical features entail distinct materials. Therefore, the use of the fraction-product described herein enables certain inferences without a test set. Application software programs may include any appropriate type of data processing software program. Blocks 102-110 of the flowchart 100 may be performed by one or more computer systems.

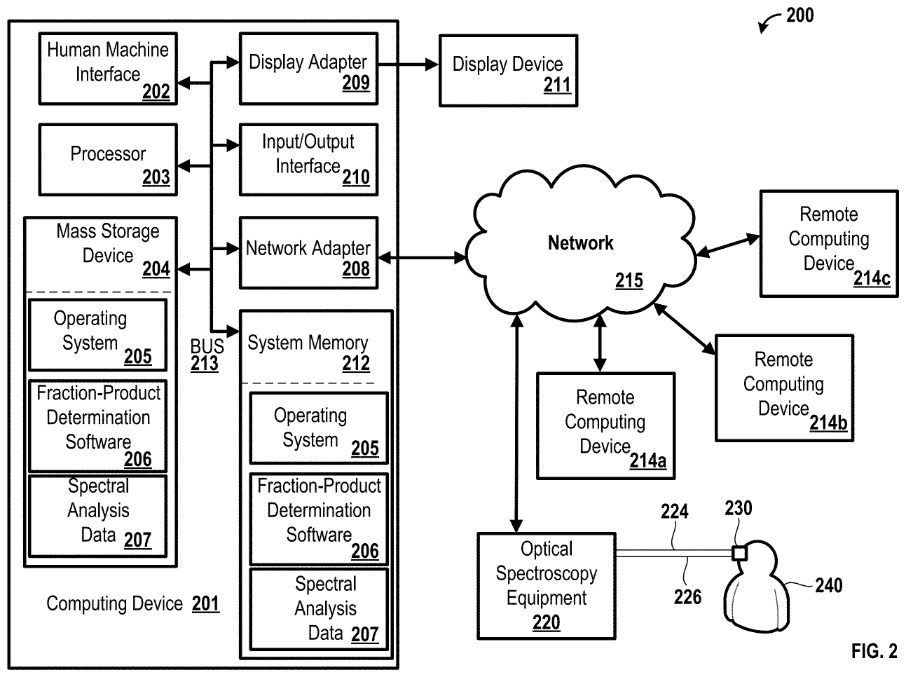

FIG. 2 shows a system 200 for evaluating brain tissue and material based on a fraction-product and, in some cases, optical spectroscopy. A computing device 201 may be used to execute steps of flowchart 100 and/or any other method, algorithm, process, and/or analysis described herein.

The computing device 201 may comprise one or more processors 203, a system memory 212, and a bus 213 that couples various components of the computing device 201 including the one or more processors 203 to the system memory 212. In the case of multiple processors 203, the computing device 201 may utilize parallel computing.

The bus 213 may comprise one or more of several possible types of bus structures, such as a memory bus, memory controller, a peripheral bus, an accelerated graphics port, and a processor or local bus using any of a variety of bus architectures.

The computing device 201 may operate on and/or comprise a variety of computer-readable media (e.g., non-transitory). Computer-readable media may be any available media that is accessible by the computing device 201 and comprises, non-transitory, volatile and/or non-volatile media, removable and non-removable media. The system memory 212 has computer-readable media in the form of volatile memory, such as random access memory (RAM), and/or non-volatile memory, such as read-only memory (ROM). The system memory 212 may store data such as spectral analysis data 207 and/or program modules such as operating system 205 and fraction-product determination software 206 that are accessible to and/or are operated on (e.g., executed) by the one or more processors 203.

The computing device 201 may also comprise other removable/non-removable, volatile/non-volatile computer storage media. The mass storage device 204 may provide non-volatile storage of computer code, computer-readable instructions, data structures, program modules, and other data for the computing device 201. The mass storage device 204 may be a hard disk, a removable magnetic disk, a removable optical disk, magnetic cassettes or other magnetic storage devices, flash memory cards, CD-ROM, digital versatile disks (DVD) or other optical storage, random access memories (RAM), read-only memories (ROM), electrically erasable programmable read-only memory (EEPROM), and the like.

Any number of program modules may be stored on the mass storage device 204. An operating system 205 and fraction-product determination software 206 may be stored on the mass storage device 204. One or more of the operating system 205 and fraction-product determination software 206 (or some combination thereof) may comprise program modules and the fraction-product determination software 206. The fraction-product determination software 206 can include multiple components that, in response to being executed by one or more processors, can perform analyses of optical spectroscopic data in accordance with aspects of this disclosure. Analysis of the optical spectroscopic data results in spectral analysis data 207. Spectral analysis data 207 may also be stored on the mass storage device 204. Spectral analysis data 207 may be stored in any of one or more databases known in the art. The databases may be centralized or distributed across multiple locations within the network 215.

Optical spectroscopic data can be acquired in vivo using optical spectroscopy equipment coupled (optically and mechanically) to an optical probe device 230 attached to the subject 240. A room where the subject is located during optical measurements can be darkened during optical spectroscopy experiments. The optical probe device 230 is herein referred to as template probe and is attached to the temple region of a subject 240. The optical spectroscopy equipment 220 can include a light source device (such as a tungsten lamp), an optical spectrograph, and a light detector device. Optical coupling between the optical spectroscopy equipment 220 and the optical probe device 230 can be provided by a source optical fiber 224 and a readout optical fiber 226. The optical probe device 230 can have multiple openings, where a first opening of the multiple openings can receive or otherwise engage the source optical fiber 224 and a second opening of the multiple openings can receive or otherwise engage the readout optical fiber 226. By selecting the first opening and second opening, a particular source-readout (or source-detector) separation can be configured. Examples of source-readout separation include 10 mm, 15 mm, 20 mm, 25 mm, and 30 mm. In this disclosure, optical spectroscopic data (or optical spectroscopy data) also can be referred to as spectroscopic data. Several subjects 240 can be probed using optical spectroscopy in accordance with aspects described herein. In some scenarios, a first group of the several subjects 240 can be afflicted by a neurological medical condition (e.g., a neuropathologic condition, such as AD) and a second group of the several subjects 240 may be a control group of subjects not afflicted by the neurological medical condition.

In some cases, the computing device 201 can obtain the spectroscopic data directly from the optical spectroscopy equipment 220. In such cases, the fraction-product determination software 206 also can include one or more components that permit controlling the acquisition of optical spectroscopic data in vivo from the subject 240. In other cases, the optical spectroscopy equipment 220 can retain the spectroscopic data obtained from the subject 240 within one or more memory devices accessible to the computing device 201. For example, such spectroscopic data can be retained within memory device(s) hosted by one or more of remote computing devices 214a,b,c. In such other cases, the computing device 201 can download or otherwise obtain the optical spectroscopic data from the one or more memory devices that retain the optical spectroscopic data.

A user may enter commands and information into the computing device 201 via an input device (not shown). Such input devices comprise, but are not limited to, a keyboard, pointing device (e.g., a computer mouse, remote control), a microphone, a joystick, a scanner, tactile input devices such as gloves, and other body coverings, motion sensor, and the like These and other input devices may be connected to the one or more processors 203 via a human-machine interface 202 that is coupled to the bus 213, but can be connected by other interface and bus structures, such as a parallel port, game port, an IEEE 1394 Port (also known as a Firewire port), a serial port, network adapter 208, and/or a universal serial bus (USB).

A display device 211 may also be connected to the bus 213 via an interface, such as a display adapter 209. It is contemplated that the computing device 201 may have more than one display adapter 209 and the computing device 201 may have more than one display device 211. A display device 211 may be a monitor, an LCD (Liquid Crystal Display), a light-emitting diode (LED) display, a television, smart lens, smart glass, and/ or a projector. In addition to the display device 211, other output peripheral devices may comprise components such as speakers (not shown) and a printer (not shown) which may be connected to the computing device 201 via Input/Output Interface 210. Any step and/or result of the methods may be output (or caused to be output) in any form to an output device. Such output may be any form of visual representation, including, but not limited to, textual, graphical, animation, audio, tactile, and the like. The display 211 and computing device 201 may be part of one device, or separate devices.

The computing device 201 may operate in a networked environment using logical connections to one or more remote computing devices 214a,b,c. A remote computing device 214a,b,c may be a personal computer, computing station (e.g., workstation), portable computer (e.g., laptop, mobile phone, tablet device), smart device (e.g., smartphone, smartwatch, activity tracker, smart apparel, smart accessory), security and/or monitoring device, a server, a router, a network computer, a peer device, edge device or other common network nodes, and so on. Logical connections between the computing device 201 and a remote computing device 214a,b,c may be made via a network 215, such as a local area network (LAN) and/or a general wide area network (WAN). Such network connections may be through a network adapter 208. A network adapter 208 may be implemented in both wired and wireless environments. Such networking environments are conventional and commonplace in dwellings, offices, enterprise-wide computer networks, intranets, and the Internet.

Application programs and other executable program components such as the operating system 205 are shown herein as discrete blocks, although it is recognized that such programs and components may reside at various times in different storage components of the computing device 201, and are executed by the one or more processors 203 of the computing device 201. An implementation of fraction-product determination software 206 may be stored on or sent across some form of computer-readable media. Any of the disclosed methods may be performed by processor-executable instructions embodied on computer-readable media.

### I. Classification of Human Neuropathology in vivo Using Near-Infrared Optical Spectroscopy

Diffuse light scattering in tissue can obscure structure within an optical spectrum, which has slowed the application of optical spectroscopy to medical diagnosis. The methods, apparatuses, computer-readable media, and systems described herein implement a novel discriminant statistic, called the “fraction-product,” that helps to discover regions of optical spectra that are useful for classifying subjects (taxonomic signal).

First-order analysis with the fraction-product reveals an optical spectroscopic feature near 861 nm that not only distinguishes those with and without a neuropathologic condition but also classifies them with 93% accuracy. The second-order analysis adds features at 677 and 809 nm. When these three limited regions of the spectrum are examined by principal component analysis, subjects are classified with 100% accuracy.

Using the fraction-product and optical spectroscopy, the methods, apparatuses, computer-readable media, and systems described herein provide the first successful classification of a human neuropathologic condition—e.g., the presence or absence of Lewy bodies as determined by autopsy—using optical spectra (e.g., near-infrared reflectance) obtained at a subject's temple while the subject is alive.

The ability to identify taxonomic signals in clinical optical spectra can enable the development of in vivo optical spectroscopy methods for neurological diagnoses, such as demonstrated here for the classification of patients based on the presence or absence of Lewy bodies in the living brain.

### A. Introduction

Following the application of near-infrared (NIR) spectroscopy to the development of pulse oximetry, in 1977 Jobsis reported the measurement in vivo of hemoglobin saturation in a cat's brain with the source fiber on one of the cat's temples and the readout optical fiber on the other one. In that paper, Jobsis also included results with the same optical configuration applied to a human head, which, being so much broader, revealed only a change in blood volume with hyperventilation. Since then most applications of NIR spectroscopy in vivo have concerned estimates of hemoglobin saturation and blood flow, although there has also been work to classify tissues biochemically and to construct images. The problem posed by the thick human head may be mitigated by placing source and readout optical fibers at the same human temple. With such a reflectance configuration, photons diffuse from the source optical fiber into the tissues and scatter back to the readout optical fiber. Time-of-flight and modeling studies confirm the notion that the greater the source-readout separation, the deeper is the mean path of the diffusing photons.

In contrast to the above-mentioned existing work, the methods, apparatuses, computer-readable media, and systems described herein determine that NIR spectroscopy can distinguish Alzheimer's disease in autopsy samples of temporal isocortex. Efforts to extend this method to living subjects have been hampered by two factors: 1) the inherent tendency of overlying tissues to scatter light, which obscures optical spectroscopic features specific to the brain, and 2) the presence of multiple neuropathologic conditions in the same individual, which create confounding taxonomic signals.

The methods, apparatuses, computer-readable media, and systems described herein mitigate the problems of tissue scattering and multiple pathologies. The emphasis is placed on identifying regions of the clinical optical spectra useful for classification, rather than on identifying underlying chemistry or morphology (extraction of which may be intractable due to the convolution of scattering and absorption in overlying tissues).

The “fraction-product” (fₚ), described herein is a novel discriminant statistic that aids in the discovery of “taxonomic signal”, that is, regions of the optical spectrum most useful for classification of a subject (or a specimen corresponding to the subject) as pertaining to a group having a neuropathological condition or a group lacking the neuropathological condition. By limiting the analysis to these regions of the optical spectrum, the methods, apparatuses, computer-readable media, and systems described herein can mitigate the tissue scattering problem. The fraction-product is applied to optical spectra from subjects selected to minimize other pathology and to isolate as the major distinguishing factor Lewy bodies, neuropathologic structures whose presence or absence is assessed routinely at autopsy. These optical spectra (data/information) can be acquired at the subject's temple while the subject is alive. The methods, apparatuses, computer-readable media, and systems described herein provide the first successful classification by NIR spectroscopy of a human neuropathologic condition in vivo.

### B. An Operational Definition of Fraction-Product fₚ

The operational definition of the fraction-product fₚ, may be described as, wherein given optical spectra of samples from two distinct populations, samples 1 and 2, at each wavelength (represented by a pixel when using a digital light detector) performing the following algorithm/steps:
1. Determine for sample 1 the median value (M₁) of the spectral feature (or optical discriminant). It is noted that each wavelength can be deemed to be an “atomic” feature that is an element of an optical feature. The optical feature is thus defined by satisfactory atomic features corresponding to respective adjacent pixels, In other words, a collection of adjacent pixels corresponding to respective atomic features forms an optical feature with linewidth.
2. Determine for sample 2 the median value (M₂) of the spectral feature.
3. Assuming that M₁ and M₂ correspond to the population values, estimate a classification cutoff as (M₁+M₂)/2. As such, M₁ and M₂ are assumed to be the median values for this data set. By using those medians as estimates of the theoretical population value, it can be asserted that members of the population not included in this data set can be classified.
4. Determine the fraction of sample 1 on the correct side of the cut-off (such fraction denoted as f₁). In other words, determined the fraction of data points of this class that are on the same side of the cut-off as their class median.
5. Determine the fraction of sample 2 on the correct side of the cut-off (such fraction denoted as f₂). In other words, determined the fraction of data points of this class that are on the same side of the cut-off as their class median.
6. Calculate the fraction-product: fₚ=f₁×f₂.

Defining the fraction-product, the algorithm/steps (steps 1-6) presuppose a paradigm in which two distinct groups are studied to devise an optical spectroscopy method (also referred to as spectroscopic method) for classifying unknowns as belonging to one group or the other. A technique for exploratory data analysis is to display the optical spectra from each group in different colors and to look for regions where the colors separate. However, regions where the colors separate may be indistinguishable to the human eye. As such, in some cases, the fraction-product may be used when distinct features may not be apparent to the eye. Accordingly, not only is the application of the fraction-product superior to techniques that rely on human intervention, but the human mind is unable to determine adequate taxonomic signals by mere visual inspection of optical spectra measured in vivo for a subject's brain.

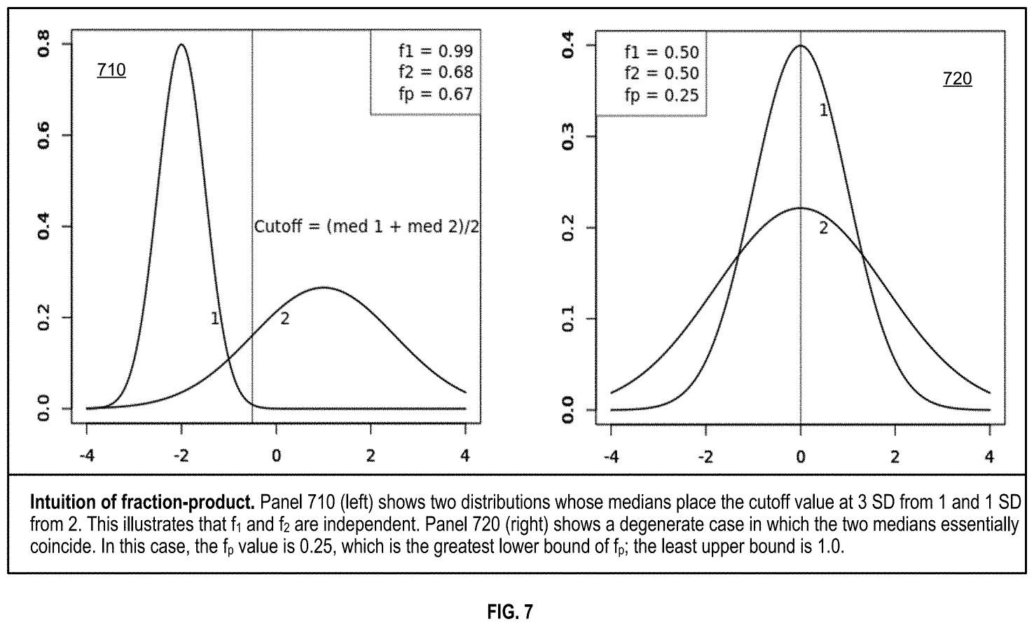

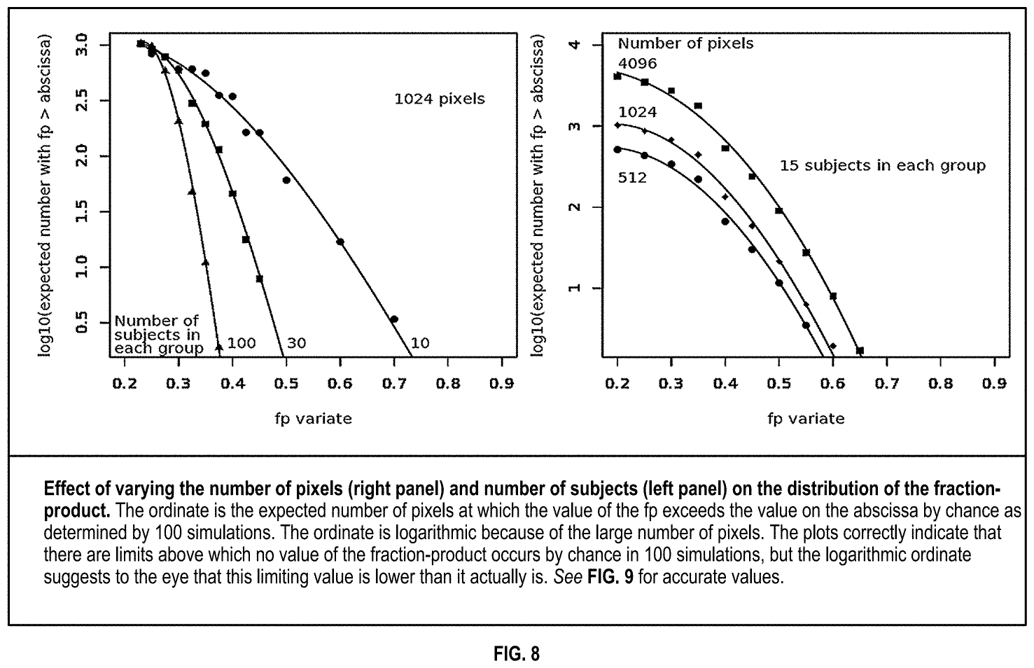

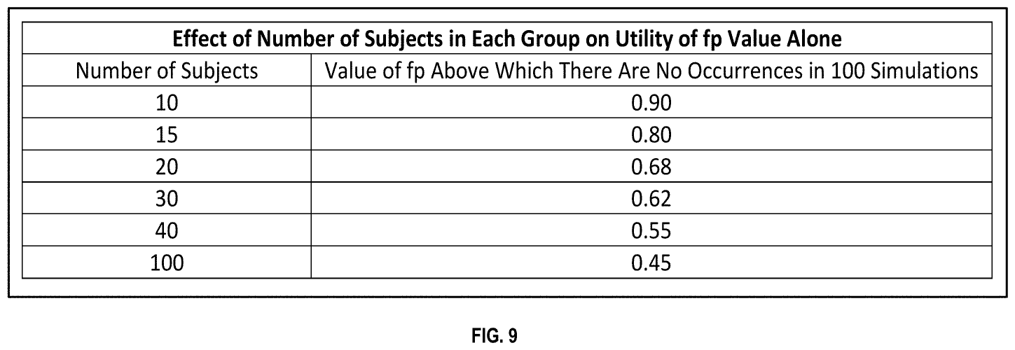

Although optical spectra are often displayed as intensities, as is used herein the term “feature” includes common transformations. For example, besides utilizing optical spectral intensity, the methods, apparatuses, computer-readable media, and systems described herein also may utilize (via the fraction-product determination software 206 (FIG. 2), for example) the first derivative of the spectral intensity curve. Assuming the use of a digital camera (e.g., a charge-coupled device (CCD) camera device), the optical spectrum for each subject may be viewed as a linear array of pixel-feature pairs. Each group (e.g., sample 1 or sample 2) is characterized by a distribution of the spectral feature measured at every pixel. The algorithm/steps described herein (steps 1-6 hereinbefore) for the operational definition of the fraction-product fₚ replace the two distributions with a respective single number. As a result, performance of such algorithm/steps generates a spectrum of values corresponding to respective fraction-products determined at each wavelength (or pixel in cases a digital light detector is used). Because the distribution of fraction-product values cannot be described by a standard mathematical formula, simulations are done to determine what results may be attributed to chance alone. In applications of the fraction-product that are described herein, the outcome is binary and the distribution of fraction-product values depends upon the number of elements in the index set (e.g., number of pixels in the spectrometer's camera) and the number of subjects in each class. As described later herein, FIG. 7 to FIG. 10D are based on simulations (SM1, SM2, SM3) that provide a more thorough exploration of the behavior of the fraction-product. Simulations SM1 to SM7 are described later herein (see section E: Simulations). FIG. 7 illustrates the intuition underlying the fraction-product. An relevant value to know is the level above which a fraction-product value has not been observed to occur in a random simulation. FIG. 9 tabulates those levels for p<0.01. Because in some embodiments, the studies disclosed herein have relatively small numbers of subjects, fraction-product values in the range from 0.7 to 0.9 can indicate effective separation of the two classes. FIG. 8 shows the effect of varying pixel number, which in some cases can be 1024, as is illustrated in at least some of the studies disclosed herein. Other simulations are matched to the number of subjects in the data analyzed.

Analysis reveals that when the two distributions at a pixel are completely separated, f₁=f₂=1 and fₚ=1. When the two distributions at a pixel essentially coincide, f₁≈f₂≈0.5, and fₚ≈0.25. Therefore, fₚ ranges from 0.25 to 1.0. Regions of the spectra where every pixel has an fₚ value close to 1 can be most useful in classifying subjects. Just as inferential statistics requires an element of judgment, e.g., choosing a level of significance, so will the criteria for choosing a threshold value of fₚ such that “fₚ>value” means “close to 1.”

Criteria to be considered are further described. Because the two groups of optical spectra are presumed to be very similar in appearance, embodiments of this disclosure can first determine (via the fraction-product determination software 206 (FIG. 2), for example) how the distribution of fₚ is affected by chance alone; useful taxonomic signals must be clearly distinct from random fluctuations. Randomness enters this paradigm through two different routes. First, there are two samples drawn from larger populations. With a small number of subjects in a sample, outliers can result in a very atypical distribution; hence, the smaller the sample size, the more random variability would be expect in the fraction-product. Second, a distribution of values from each group occurs at each pixel. Random fluctuation at each pixel may be viewed, for the sake of explanation, as drawing balls from two Gaussian urns, and, the more pixels, the greater the opportunity for a rare event to occur. As described later herein, simulations (SM2) to determine how changes in the number of pixels and the number of optical spectra (e.g., number of subjects) affect the utility of the fraction-product have been performed. These simulations treat two adjacent pixels as if the distributions at each are completely independent. However, real optical features have linewidth and extend over several contiguous pixels; applying this fact to the interpretation of the fraction-product greatly increases its utility. Once the threshold value to determine “close to 1” has been chosen, then the probability that adjacent pixels are “close to 1” appears as the probability of success in a Bernoulli process.

Two important results of the simulations described later herein are the following: (1) for smaller numbers of subjects (N<15), a random simulation matching the physical experiment in pixel number and subject number must be done for comparison; (2) although the value of fₚ alone may be significant for N>15, the occurrence of values of fₚ close to 1 on contiguous pixels (linewidth) is far more useful for discovering taxonomic signal. Given these results, optical spectroscopic data are compared to a random simulation with corresponding numbers of subjects and pixels.

### C. Application of the Discriminant Algorithm to Subjects With and Without Lewy Bodies

In collaboration with the Boston University Alzheimer's Disease Center, the use of NIR spectroscopy following the methods, apparatuses, computer-readable media, and systems described herein can be used as a tool for understanding neurodegenerative disease. As described later herein, SM3 contains a detailed description of the spectroscopic methods and subject demographics. In some embodiments, light from a tungsten lamp passes through an optical fiber to the subject's temple. For the measurements discussed herein, the readout optical fiber (e.g., readout optical fiber 226 (FIG. 2)) is placed on the same human temple 25 mm from the source optical fiber (e.g., source optical fiber 224 (FIG. 2)) and conducts the light to an imaging spectrograph that disperses incoming light from 644 nm to 1052 nm over 1024 pixels. Spectra are corrected for lamp spectrum and integration time and are then normalized to unit area and smoothed. The first derivative of the intensity spectrum is used in this analysis; the resultant numbers are referred to as slope variates, and those from both temples are averaged before statistical analysis. With the readout optical fiber placed 25 mm from the source fiber, the light field interrogated includes a portion of the superior temporal gyrus as well as the overlying cerebrospinal fluid, skull, and scalp.

All subjects were patients in the Dementia Special Care Unit of the Edith Nourse Rogers Memorial Veterans Hospital enrolled in a protocol approved by the institutional review board (IRB). All subjects reported came to autopsy, and Lewy bodies were demarcated by anti-synuclein antibodies. In the setting of advanced dementia, two points of background information come into play. First, as mentioned, it is expected that each subject likely has multiple neuropathologic findings, minimally those of Alzheimer's disease and vascular disease. Second, it is a principle of neuropathology that lesions in one area of the brain can cause changes in other areas through transneuronal degeneration. To isolate the presence of Lewy bodies as a factor and to address the issue of multiple pathologies, those subjects with frontotemporal lobar degeneration and obvious infarcts were eliminated. Further control for variations in Alzheimer's pathology was employed by selecting those subjects with the same classification, “high likelihood” by the Reagan criteria. In this selected subset, there are 7 spectra in the group without Lewy bodies and 9 spectra in the group with Lewy bodies, 2 with Lewy bodies in the temporal isocortex probed by the light field in our measurements, and 7 with Lewy bodies elsewhere in the brain. It is reasonable to assume that in advanced dementia, sufficient time has elapsed for transneuronal degeneration effects, if any, to have taken place in the temporal lobe due to Lewy bodies outside it. Therefore, to avoid confounding the effects of transneuronal degeneration with those due to the physical presence of Lewy bodies, the 2 subjects with Lewy bodies in the temporal isocortex are excluded from the analysis. The effect of removing these two subjects is discussed below and later herein (SM8).

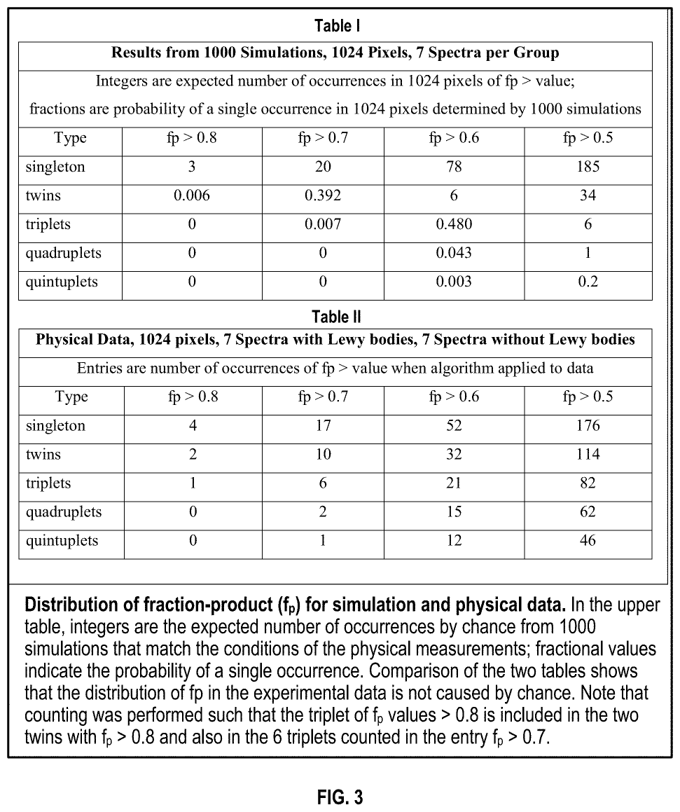

To assess what range values of fₚ can assume by chance alone under experimental conditions, 1000 simulations were performed with the numbers of subjects and pixels matching those of the physical experiment and the values analogous to the slope variates generated randomly. FIG. 3 shows the results. To describe occurrences in which fₚ exceeds a particular value on adjacent pixels, the terms twins, triplets, etc. are used. The fraction-product determination software 206 (FIG. 2) that assesses adjacent pixels proceeds sequentially and tests for fₚ>threshold value; therefore, it counts a triplet also as two twins and three singletons and similarly for the quadruplets and quintuplets. As expected, the higher the selected value of fₚ, the less likely it is to occur by chance; even more so, the higher the selected value of fₚ, the less likely will three or four such values occur on contiguous pixels. If, for example, the first entry in the column fₚ>0.5 for simulations (FIG. 3) is used to estimate the probability of success (185/1024=0.1807), then the subsequent entries for twins, triplets, etc., closely follow a Bernoulli process (p<10⁻⁴; linear model), and similarly for fₚ>0.6 (78/1024=0.0762; p<10⁻⁵; linear model). Comparing the distributions of fₚ from the physical data to the simulations shows clear differences. In the physical data, if fₚ>0.8 is taken as “close to 1,” there is a triplet (see bottom table in FIG. 3), the occurrence of which has an estimated p-value of (4/1024)³≈10⁻⁸.

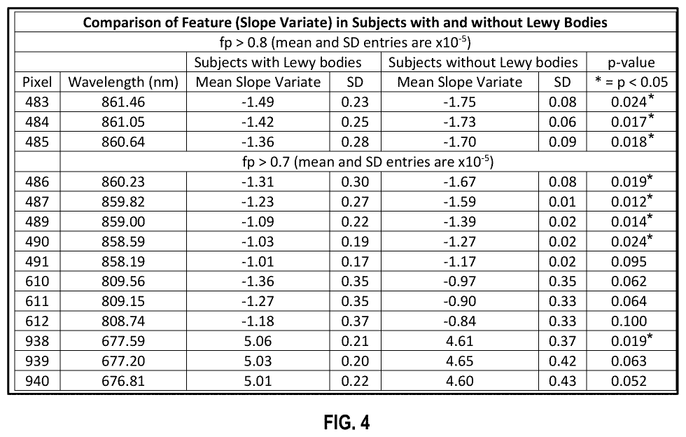

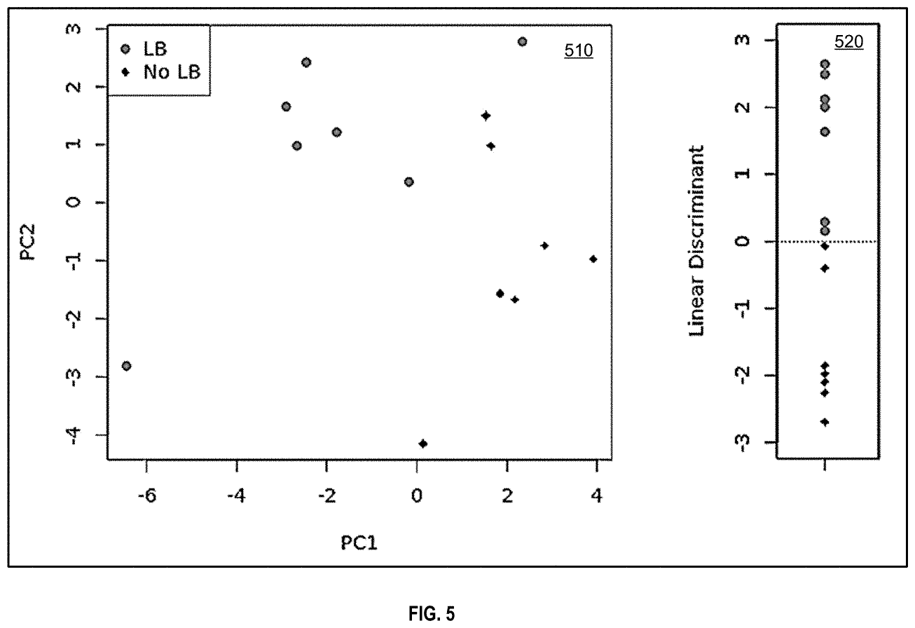

FIG. 4 shows a summary of statistics for fₚ>0.8. At each one of the three wavelengths (861.46 nm, 861.05 nm, and 860.64 nm), a cutoff may be chosen such that at most one subject is misclassified (93% accuracy). Furthermore, by Student's t, the slope variate at each of these wavelengths measures a statistically significant difference between those subjects with and without Lewy bodies. If fₚ>0.7 is chosen as a notion of “close to 1,” then there are three triplets and one quintuplet, none of which could be attributed to chance (FIG. 3). These were centered around 860 nm (quintuplet and one triplet), 809 nm (triplet), and 677 nm (triplet); one member of the triplet at 677 nm was a singleton for fₚ>0.8. These regions of the whole optical spectrum were merged into a limited 14-pixel subset, on which principal component analysis (PCA) can be performed using the fraction-product determination software 206 (FIG. 2) or, in some cases, the prcomp function of R with mean-centering and scaling to unit variance. The results are shown in FIG. 5 (panel 510). SM4 discusses the selection of “close to 1.”

### Principal Component Analysis and Linear Discriminant Analysis

FIG. 5 shows the results of principal component analysis and linear discriminant based on the methods, apparatuses, computer-readable media, and systems described herein. The first two principal components (PC1 and PC2; panel 510 in FIG. 5) display a clear separation of the subjects with Lewy bodies (gray circles) and without Lewy bodies (black diamonds). PC1 and PC2 contain 79% of the total variance. Application of linear discriminant analysis to PC1 and PC2 results in complete separation of the two groups (right panel).

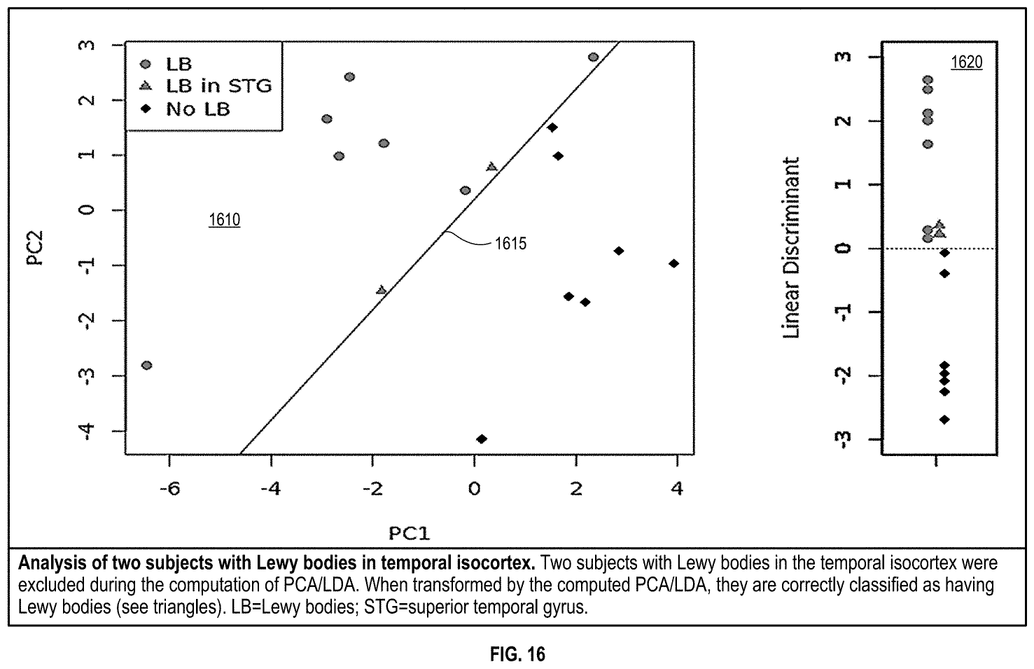

Linear discriminant analysis (LDA) can be performed on the first two principal components using the fraction-product determination software 206 (FIG. 2) or, in some cases, the LDA function of R and reducing the data to one dimension (panel 520 in FIG. 5). There is a complete separation between the two groups (p<10⁻⁴). If the two subjects with Lewy bodies in the temporal isocortex, which were not included in the analysis, are input as unknowns into the PCA/LDA transformation, they are classified correctly (SM8, FIG. 16).

### D. Discussion

In principle, when light from a single source illuminates two distinct materials, the resultant spectra will be distinct, a priori. The fraction-product enables the discovery of the optical spectroscopic features responsible for such distinctions.

Eight optical spectroscopic slope variates (starred entries in the p-value column in FIG. 4) physically distinguish those subjects with and without Lewy bodies. By combining three sub-regions of the spectra comprising these variates for principal component analysis, subjects with and without Lewy bodies are completely differentiated. The ability to classify subjects according to some pathophysiological factors, such as Lewy bodies, can be a prerequisite to developing any diagnostic test while identifying the underlying biophysical origins of the distinguishing optical spectroscopic features continues to be of great interest.

Based on the methods, apparatuses, computer-readable media, and systems described herein, the fraction-product can be used (via the fraction-product determination software 206 (FIG. 2), for example) as a discriminant statistic to discover potentially useful regions of similar spectra, that is, regions containing a taxonomic signal. However, current cameras have millions of pixels, so unlikely events occur, which might create a false impression of a useful optical spectroscopic region. Accordingly, the fraction-product and the assumption of a Bernoulli process provide a basis for inferring whether triplets, etc. have occurred by chance or represent a taxonomic signal. This inference is separate from whether the selected regions (which serve as classifiers) lead to a statistically significant difference between the two groups.

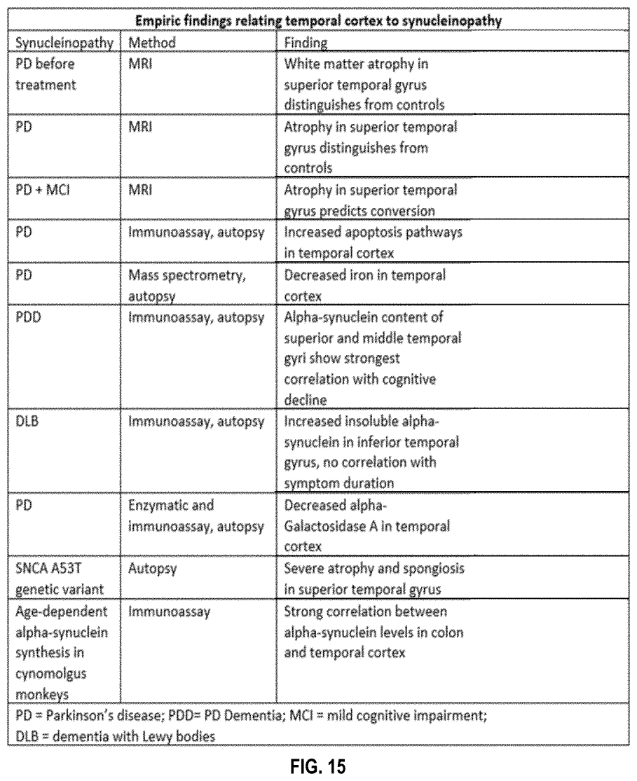

Lewy bodies were originally described as eosinophilic, proteinaceous structures that were later associated with Parkinson's disease. Since alpha-synuclein was determined to be a major protein component of Lewy bodies, a more general concept of synucleinopathies has emerged, which also includes dementia with Lewy bodies and multiple system atrophy. It has been recognized for well over a century that neurodegeneration starting in one location can affect distant parts of the brain. The staging of Parkinson's disease proposed by Braak and colleagues mentions that the pathology in Stage 6 can affect the superior temporal gyrus; therefore, the region probed by our light field includes tissue known to be affected by the most common synucleinopathy. Other studies that document the effects of synucleinopathy on the temporal cortex are summarized in FIG. 15. While it may not be asserted definitively that reported optical spectroscopic differentiation of subjects with and without Lewy bodies arises in the superior temporal gyrus, it is highly unlikely that the overlying tissues are affected by Lewy bodies in the brain.

Several observation can me made regarding the results yielded by the systems and methods of this disclosure. First, the successful classification is due in part to the elimination of subjects with other significant pathology such as infarcts and frontotemporal lobar degeneration. The rapid discovery of potentially useful regions of the spectra by the fraction-product leads to optimism that the extension to more complicated cases will be attainable. Second, the number of subjects involved is small. However, p-values for the features around 861 nm and 677 nm indicate statistical significance, and the correct classification of two subjects with Lewy bodies in the temporal isocortex that were not included in the determination of the principal components (see SM8) also supports the expectation that the method will apply to larger samples. The ability to extend this non-invasive approach to studies of the general population underscores its potential for use in clinical screening and, ultimately, diagnosis.

### E. Simulations

### Simulations 1 (SM1)—The Fraction-Product in the Analysis of Lewy Bodies

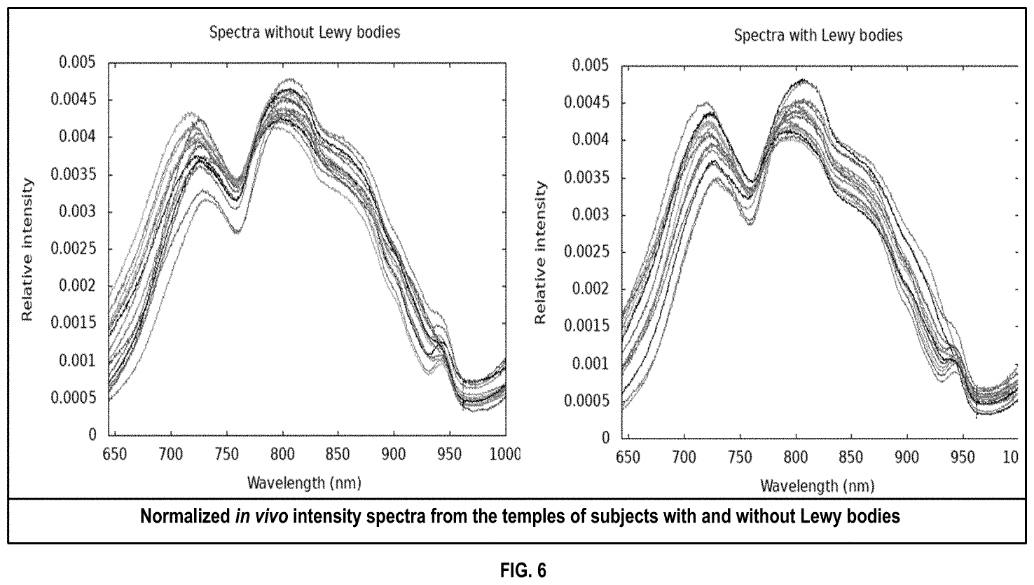

In this analysis, taxonomic signal was searched between one group that had Alzheimer's disease without Lewy bodies (the group consisting of 7 subjects) and another group that had Alzheimer's disease and Lewy bodies in areas of the brain outside the temporal isocortex (that other group also consisted of 7 subjects). As it can be gleaned from FIG. 6, there is no stark or otherwise obvious difference between the intensity spectra of the two groups. The systems, apparatuses, computer-readable media, and methods of this disclosure can apply (via the fraction-product determination software 206 (FIG. 2), for example) the fraction-product to search for taxonomic signal in the first derivatives of the spectra in FIG. 6. Because in some cases there are 1024 pixels in the light detector utilized in the optical spectroscopy measurements, many of the significant fₚ values are expected to be due to random fluctuation. Therefore, Simulation 1 (SM1; summarized at Table I in FIG. 3) shows the results of fₚ values due to randomness under these conditions. For example, the actual data (Table II in FIG. 3) shows that one set of three adjacent pixels (a triplet) all had fₚ>0.8. In 1,000 random simulations there were no such triplets (see Table I in FIG. 3), indicating that the probability that the data triplet was due to chance is<0.001. Therefore, from the analysis performed using the fraction-product, the wavelengths corresponding to those three pixels emerge as a very strong candidate to distinguish Lewy bodies.

As mentioned, when light from the same source illuminates two distinct materials, the two resultant spectra are distinct a priori. In practice, especially in biological systems, spectra from different specimens may appear to be so similar as to be indistinguishable. In the instant case, it is unknown in advance whether the parenchyma of the superior temporal gyrus would reveal whether Lewy bodies were present elsewhere in the brain; however, based on exploratory data analyses and the classical process of transneuronal degeneration, it was expected that the parenchyma would differ (see FIG. 15). The problem is to find where the optical spectra differed.

FIG. 7 shows the general behavior of the factors that affect the value of the fraction-product. In principle, the two fractions f₁ and f₂ are independent. In FIG. 7, “SD” refers to standard deviation.

### Simulations 2 (SM2)

To assess the taxonomic signal using the fraction-product, it is first determined how the variate's distribution is affected by chance alone. As mentioned, randomness enters this experimental paradigm through two different routes. First, via two groups that have been drawn from larger populations. Second, a distribution of values from each group occurs at each pixel, and the more pixels, the greater the opportunity for a rare event to occur. Simulations were used to determine how changes in the number of pixels and the number of optical spectra (or number of subjects, in some cases) affect the utility of the fraction-product. As also mentioned, these simulations treat two adjacent pixels as if the distributions at each are completely independent. However, real optical features have linewidth and extend over several contiguous pixels. Applying this fact to the interpretation of the fraction-product greatly increases its utility. FIG. 8 shows the effect of varying the number of pixels (right panel) and the number of subjects (left panel) on the distribution of the fraction-product.

Simulations can be performed using the fraction-product determination software 206 (FIG. 2) or, in some cases, statistical analysis software such as R. Every simulation required as input a given number of pixels for each optical spectrum and numbers N1 and N2 defining the number of optical spectra in respective groups. In some cases, the number of optical spectra coincides with the number of subjects in a group. At each pixel, N1 and N2 values were randomly generated from the same unit normalized distribution and the fraction-product was determined in accordance with aspects described herein. For all simulations disclosed here, N1=N2. One hundred simulations were performed for each set of inputs. As described, FIG. 8 shows the effects of varying the number of pixels and the number of optical spectra. Because of the large number of pixels, the logarithm base 10 of the number is plotted as ordinate. Although this distorts the shape of the curve, it visually reveals the fact that, for each set of conditions, there is a value of the fraction-product above which there is no random occurrence. Because there were 100 simulations, the probability of a random occurrence is less than 0.01. These values are summarized in FIG. 9. The practical significance of these numerical facts is that, for example, if an experiment with 15 or more subjects generated a fraction-product value greater than 0.85, it is very unlikely to be due to chance and hence more likely indicates a region of the spectra that distinguishes the two groups. That is, the region contains taxonomic signal.

### Simulations 3 (SM3)—Behavior of the Fraction-Product for Small Sample Sizes

Because the estimated cut-off is the average of the two medians, the values of f₁ and f₂ must be greater than 0.5. Therefore, for small sample sizes, the fₚ can take on only a few values. For example, if the two groups have only 5 subjects each, then f₁ and f₂ can assume the values 0.6, 0.8, and 1.0 and fₚ can take on one of the six values: 0.36, 0.48, 0.6, 0.64, 0.8, 1.0. When the number of subjects is 10 or more, the range of fₚ is fine enough that this discreteness is no longer a significant issue. Because of this behavior, for small N it is best to perform simulations with the exact numbers of subjects if N<10. For the data presented here, the results of the simulation for N1=N2=7 are given in FIG. 3.

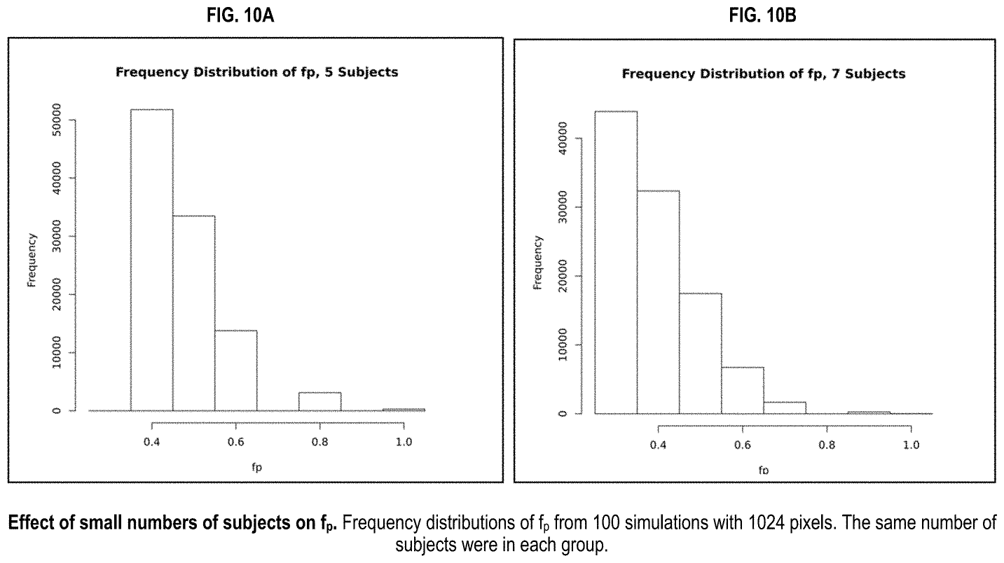

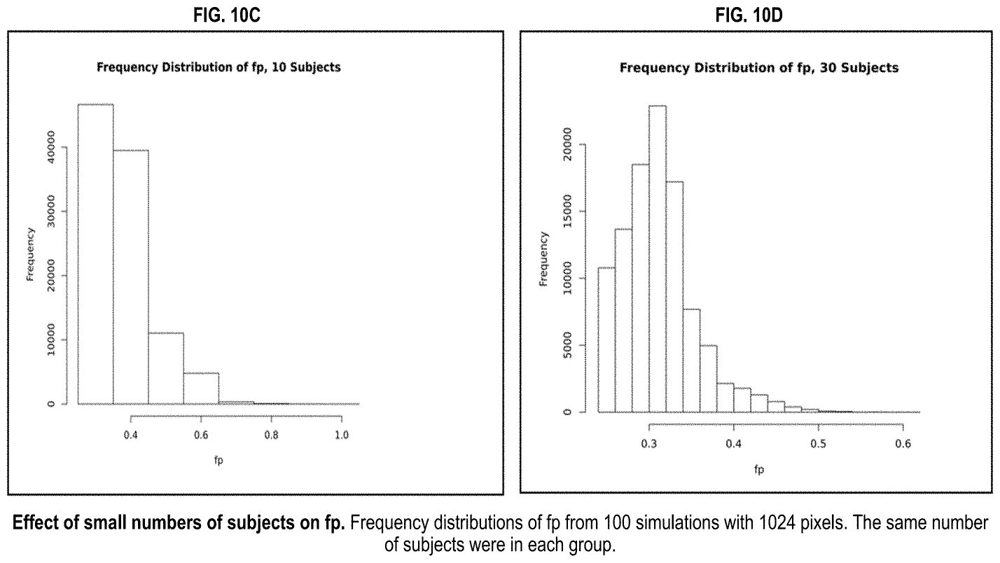

FIG. 10A to FIG. 10D provide more detailed behavior for simulations with small numbers of subjects. Particularly, FIG. 10A to FIG. 10D show the effect of small numbers of subjects on fₚ. Frequency distributions of fₚ from 100 simulations with 1024 pixels. The same number of subjects were in each group. Although the discreteness of fₚ is apparent in the frequency distribution for 5 subjects (FIG. 10A) in each group, it is much less apparent for 10 subjects (FIG. 10C). Note that the binning is the same for these two histograms. At 30 subjects in each group (FIG. 10D), the distribution resembles that of a continuous random variable.

This behavior might limit the utility to use only the value of fₚ to assess how likely is a result due to chance. However, it has little impact on using contiguous pixels with fₚ above a particular value as an indication that the result is not due to chance.

### Simulations 4 (SM4)—Application of the Fraction Product

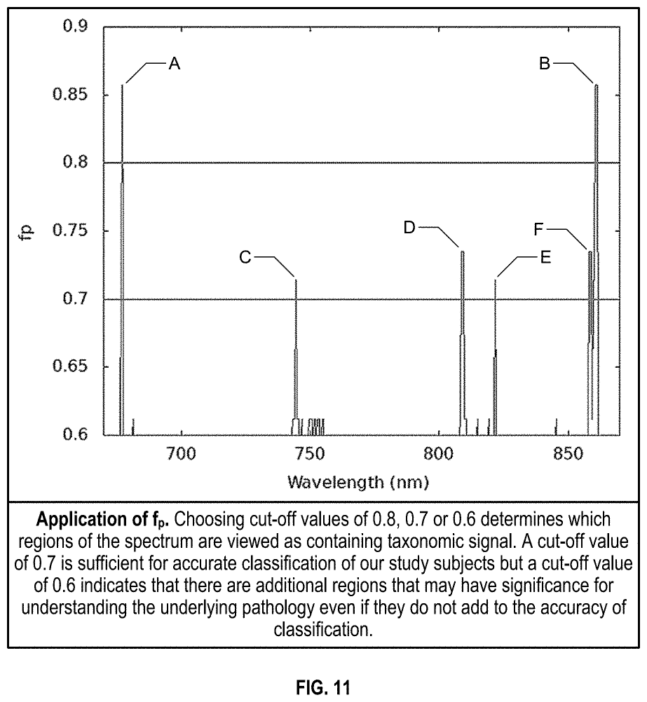

The utility of the fraction product lies in its ability to find a taxonomic signal. As with any other inferential statistic, there is flexibility in the selection of cut-offs for significance. FIG. 11 shows how choosing cut-offs of 0.8, 0.7, and 0.6 for fₚ includes increasingly greater regions of the spectrum for consideration. A cut-off of 0.8 includes a region A and a region B. A cut-off of 0.7 adds a region C, a region D, a region E, and a region F, yielding a total of six total regions of interest (or candidate regions). As described in the caption to FIG. 11, the previous stop was at 0.7 because the goal of accurate classification was achieved. FIG. 11 reveals that with 0.6 as a cut-off, additional regions of taxonomic signals appear, which could have scientific interest even if they do not improve classification.

### Simulations 5 (SM5)—Subject Demographics

Subjects were recruited as part of an ongoing project to monitor the progression of neurodegenerative diseases, especially Alzheimer's disease. All subjects were recruited through a process approved by the Institutional Review Board; informed consent was obtained in all cases. Subjects clinically diagnosed with senile dementia of the Alzheimer's type were recruited from the inpatient dementia unit of the Edith Nourse Rogers Memorial Veterans Hospital. All autopsies were performed by one neuropathologist.

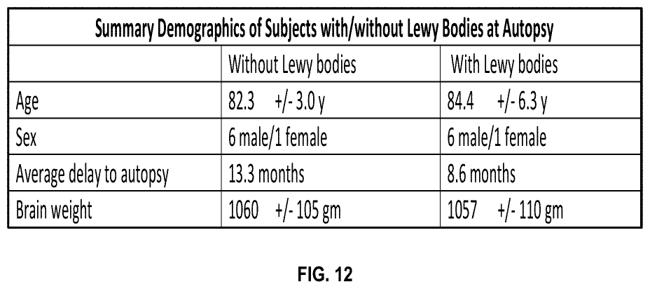

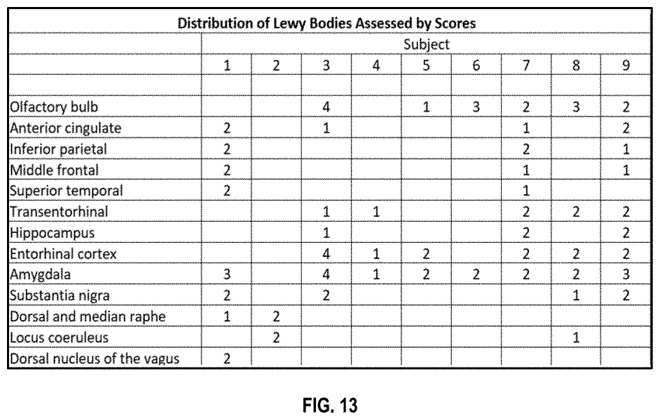

General demographic information is summarized in FIG. 12. Age refers to that at the time of measurement; the distribution of sexes is skewed by the population of Veterans in the hospital. Measurements were made in two sessions, about two years apart. Two subjects were measured in both sessions and, for them, “average delay to autopsy” refers to the second measurement. The two subjects with Lewy bodies seen in the temporal neocortex were both males, aged 76 and 83 at the time of measurement. FIG. 13 shows the results. Other potential factors that were examined included Braak stage for Alzheimer's disease and the degree of amyloid angiopathy, arteriosclerosis, and atherosclerosis; there was some heterogeneity but no systematic differences between the two groups. None of the differences between the two groups reached statistical significance at the p<0.05 level. In other words, the presence or absence of Lewy bodies is being classified, not one of these other factors. Lewy bodies were assessed by the anti-synuclein antibody as previously described. Lewy neurites were noted but always occurred with Lewy bodies and hence have not been mentioned.

### Simulations 6 (SM6)—Near-Infrared Reflectance Measurements

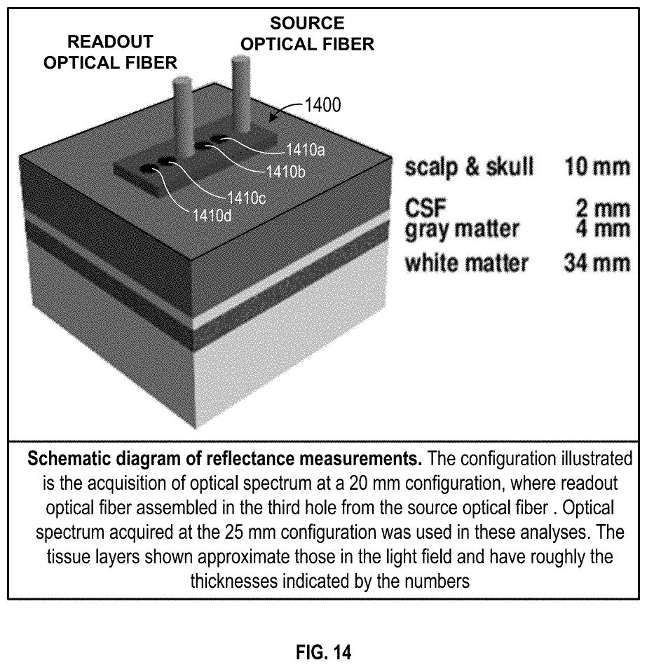

In some embodiments, optical measurements can be performed through fiber optic cables made of silica with a low hydroxyl concentration, with a diameter of 600 μm (source optical fiber) and 200 μm (readout optical fiber) and with a numerical aperture (NA) of 0.22. The disclosure is not limited to optics having such attributes. Other optics can be utilized to collect spectroscopic data in vivo in accordance with aspects of this disclosure. Regardless of the optics utilized to collect the spectroscopic data in vivo, the spectroscopic data can be supplied to a computing device (e.g., computing device 201 (FIG. 2) for analysis in accordance with aspects described herein. FIG. 14 shows a schematic diagram of a plastic template probe 1400 constructed by drilling one hole for the illuminating fiber (or source optical fiber) and five holes at distances from 10 mm, 15 mm, 20 mm, 25 mm, and 30 mm from the hole engaging the source optical fiber, for the various positions of the readout optical fiber. The source optical fiber and the readout optical fiber shown in the plastic template probe 1400 can embody, respectively, the source optical fiber 224 (FIG. 2) and the readout optical fiber 226 (FIG. 2). The five holes include a hole 1410a placed at 10 mm from the source optical fiber; a hole 1410b placed at 15 mm from the source optical fiber; a hole placed at 20 mm from the source optical fiber (shown as engaging the readout optical fiber); a hole 1410c placed at 25 mm from the source optical fiber; and a hole 1410d placed at 30 mm from the source optical fiber. The template probe 1400 was secured to the temple by an elastic band around the subject's head. The template probe 1400 can embody, or can constitute, the optical probe device 230 (FIG. 2). The readout optical fiber conducted the reflected light to an imaging spectrograph (Kaiser Optical Systems, Ann Arbor, Mich.) using a camera cooled to −50° C. (Andor Technologies, South Windsor, Conn.) as light detector device. Two lamps, a 7 W and a 20 W tungsten lamp (Ocean Optics, Dunedin, Fla.), were used for the first and second sessions respectively. The disclosure is not limited to such template structure (or template probe) and/or spectroscopy equipment.

At each source-readout separation two spectra were collected, one at each temple. The 25 mm source-detector separation spectra were analyzed. One reason to analyze such spectra is that such a separation is consistent with theory on light propagation in the human head indicating that at this source-detector separation the detected light has propagated through the temporal cortex, which is a site of early and extensive Alzheimer disease involvement. Each spectrum was corrected for acquisition time and background; correction for lamp output and detector response was achieved by a reference spectrum obtained by reflection from barium sulfate (first session) or a Spectralon low reflectance standard (Labsphere, North Sutton, N.H.) (second session). The average spectrum of the two temples was used for data analysis. To calculate the first derivative, the optical spectrum can be smoothed by boxcar averaging and the slope computed as a least-squares fit of a straight line through a region spanning n=11 pixels. The fraction-product determination software 206 (FIG. 2) can determine the first derivative in such a fashion, in response to being executed by one or more processors in a computing device (e.g., computing device 201 (FIG. 2)). Other values of n can be used in the determination of the first derivative.

It is noted that between the two sessions, there were different light sources, readout optical fiber cables, and methods of correcting for the reference spectrum. These differences support the conclusion that the results truly depend upon the acquired spectra rather than the technical features of the equipment used.

Simulations 7 (SM7)—Experimental Evidence for an Effect of Synucleinopathies on the Temporal Cortex

As previously described, it is a fundamental principle of neuropathology that disease in one area of the brain can lead to changes in other areas. Nonetheless, in the case of synucleinopathies, there is also empirical evidence that such effects occur in the temporal cortex regardless of where the pathology may be. FIG. 15 shows a summary of empiric findings relating to synucleinopathy.

### Simulations 8 (SM8)—Classification of Two Subjects Omitted from the Computation of PCA/LDA

Two subjects with Lewy bodies in the temporal isocortex were omitted from PCA/LDA so as not to confound the optical effects of actual Lewy bodies with those from transneuronal degeneration. When the optical spectra from these two subjects are analyzed (via the the fraction-product determination software 206 (FIG. 2), for example) as unknowns and plotted after being transformed by the previously computed PCA and LDA, they are correctly classified as is shown in FIG. 16. More specifically, spectroscopic data observed for those two unknown subjects lies in a “Disease” region in principal-component space, as defined by the decision line 1615. See triangles in FIG. 16. This finding strengthens the conclusion that these results extend to larger datasets.

### II. Alzheimer's Pathology Through the Near-Infrared Window

Near-infrared reflectance spectroscopy may be applied to the human temple. Using feature selection on the first derivative of the normalized optical intensity spectrum, regions around 860 nm and 895 nm that separate subjects who have autopsy-confirmed Alzheimer's disease without significant other pathology from age-matched controls may be determined. Principal component analysis demonstrates that these two wavelengths (or features) also separate mildly cognitively impaired subjects according to the degree of impairment. Linear discriminant analysis reveals that the 895 nm feature plays a greater role in separating mildly impaired subjects from controls (ratio of weights: 1.3), whereas the 860 nm feature is more important for distinguishing mildly impaired from Alzheimer's disease (ratio of weights: 8.2). Clinical trials may be used to validate/confirm the two features as useful for tracking disease progression and may be used to monitor potential therapeutic interventions early in the course of Alzheimer's disease.

Alzheimer's disease touches millions of families throughout the world and severely burdens all those affected. Research has improved understanding of the cellular pathology of the protein tau and amyloid precursor protein, which answer to the pathognomonic lesions: tangles and plaques. However, little progress has been made towards therapy because the insidious onset of symptoms masks the ongoing irreversible damage until a diagnosis can be established, a situation that leads to the stark observation that “Everyone knows a cancer survivor. Nobody knows an Alzheimer's survivor.”

The methods, apparatuses, computer-readable media, and systems described herein may be used to assess the effect of early interventions, which could greatly accelerate the development of effective treatment and prevention. Two features of near-infrared reflectance spectra, for example, acquired at the temple, can distinguish subjects with Alzheimer's disease from normal, age-matched controls. Moreover, these two features can classify subjects with mild cognitive impairment according to the degree of severity. This novel approach to analyzing the optical spectra may be applied in other areas, such as drug development, materials design, and semiconductor processing and packaging just to name a few examples. Clinical trials may be used to assess the utility of the methods, apparatuses, computer-readable media, and systems described herein.

Although most applications of near-infrared spectroscopy to the human head involve oximetry or blood flow, more recent advances have included imaging and co-registration studies. In contrast, the methods, apparatuses, computer-readable media, and systems described herein use optical spectroscopy as a non-invasive method to detect Alzheimer's disease (AD) that is suitable for widespread screening. It is demonstrated that reflectance spectroscopy distinguishes autopsy samples from brains with AD from those without. The approach is extended to living subjects by utilizing the relative transparency of biological tissue to light in the 700-1100 nm range, a region known as the near-infrared window. A standard reflectance configuration may be used with the optical fibers from the source with the same temple placed 25 mm apart from the readout optical fibers that transport reflected signal to a detector. With this configuration, the light field interrogated includes a portion of the superior temporal gyrus in addition to the overlying tissues.

A general principle, often left unstated, that motivates applications of optical spectroscopy to medical diagnosis is: if the same light source illuminates two distinct materials, the resultant optical spectra are a priori distinct. However, the scattering of light from biological tissues often renders it extremely difficult to discover spectral features that mark the difference. To mitigate this problem, algorithms from the field of feature selection are used to search for those regions of the optical spectra that best distinguishes two groups. Optical spectra from dementia subjects were acquired while they were alive, and only those for whom post mortem examination confirmed the diagnosis of AD were used. Autopsy reports raised another well-known problem: the brains of the elderly often have multiple morbidities such as infarcts and Lewy bodies. To mitigate this problem, seven subjects with AD (NIA-Reagan: high likelihood; Braak neurofibrillary stage: VI) but no other significant pathology were identified—the closest to “pure AD” in practice. Control subjects who volunteered for the Boston University Alzheimer's Disease Center's HOPE protocol, which evaluates the cognitive function of its subjects annually, were used. In some embodiments, the feature used was the first derivative of the normalized optical spectral intensity as a function of wavelength. This feature is herein referred to as the slope variate.

### A. Results

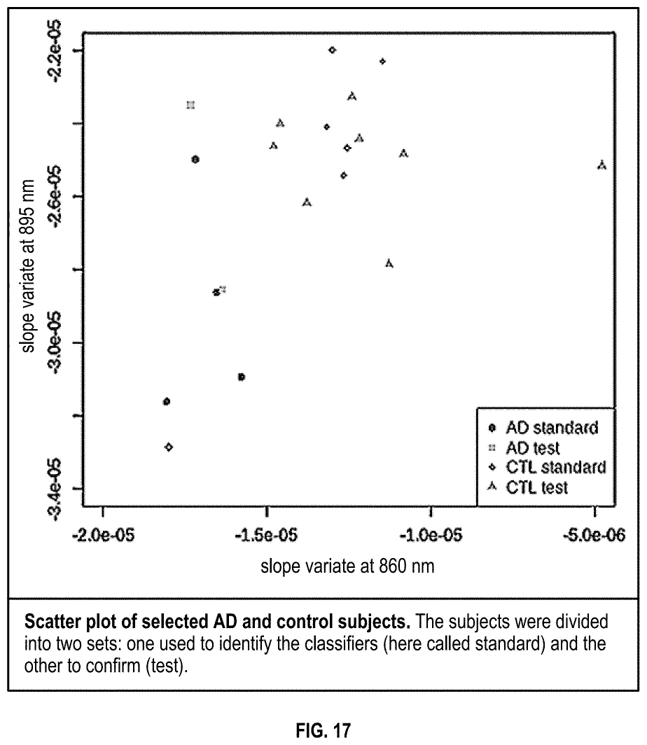

Two feature selection algorithms can be applied to spectroscopic data corresponding to the pure AD and control groups. The fraction-product determination software 206 (FIG. 2) can implement the two feature selection algorithms in accordance with aspects described herein. More specifically, the fraction-product determination software 206 (FIG. 2), in response to being executed by one or more processors of a computing device (e.g., computing device 201 (FIG. 2) can implement such algorithms. One of the applied algorithms uses the mean, and the other one of the applied algorithms uses the median. Both feature selection algorithms yielded similar results. Two candidate regions emerged: one centered around 895 nm (spanning 3 pixels; a triplet in the nomenclature of FIG. 3), the other around 860 nm (spanning 4 pixels; a quadruplet in the nomenclature of FIG. 3). Linear discriminant analysis was applied to these 7 pixels and determined that the slope variates at 895.68 nm and 860.64 nm contributed the most to distinguishing AD from control. A scatter plot of these two variates (pixels) is shown in shown in FIG. 17. This process may be considered a two-point calibration (pure AD and control). Feature selection includes separating the data into two groups, one group used to determine the best feature (a discovery set), the other group used to confirm (a test set). FIG. 17 shows distinctions between the subjects used in discovery (5 AD and 5 controls as standards) and those used to confirm (2 AD and 8 controls as tests). The two features are uncorrelated (Pearson's product-moment correlation: 0.13 pure AD; −0.15 controls). AD and control points fall within two different regions of FIG. 17, which is most useful for classification. Furthermore, their separation is statistically significant (p<0.001; Hotelling's T2-test), a fact which is not guaranteed by optimal feature selection.

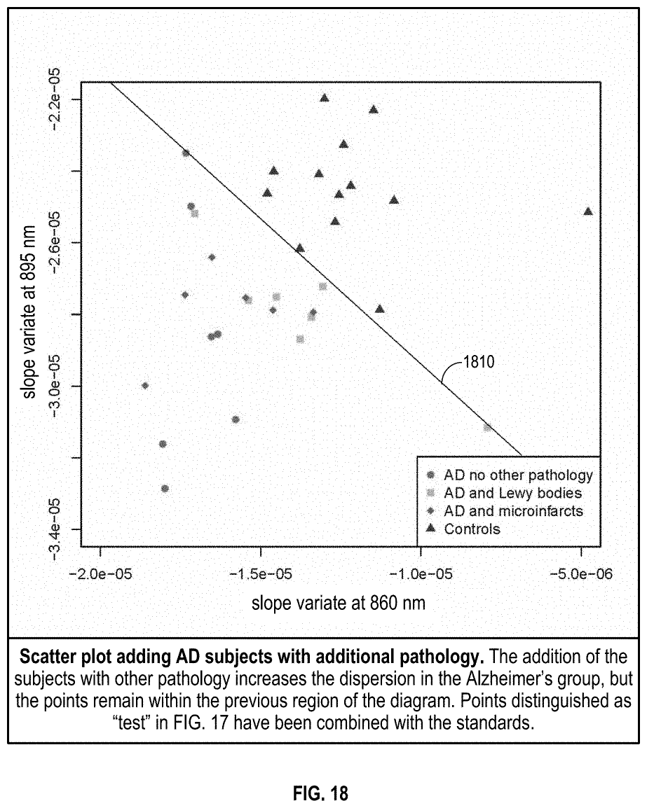

FIG. 18 shows the addition of those autopsy subjects that also had a high likelihood of AD by the NIA-Reagan criteria but had significant other pathology: infarcts (6 subjects) or Lewy bodies (7 subjects). Clearly, the dispersion of the data points increases but they remain within the two general regions of the plot that distinguished pure AD from control. The separation of the two groups remains statistically significant (p<0.001; Hotelling's T2-test).

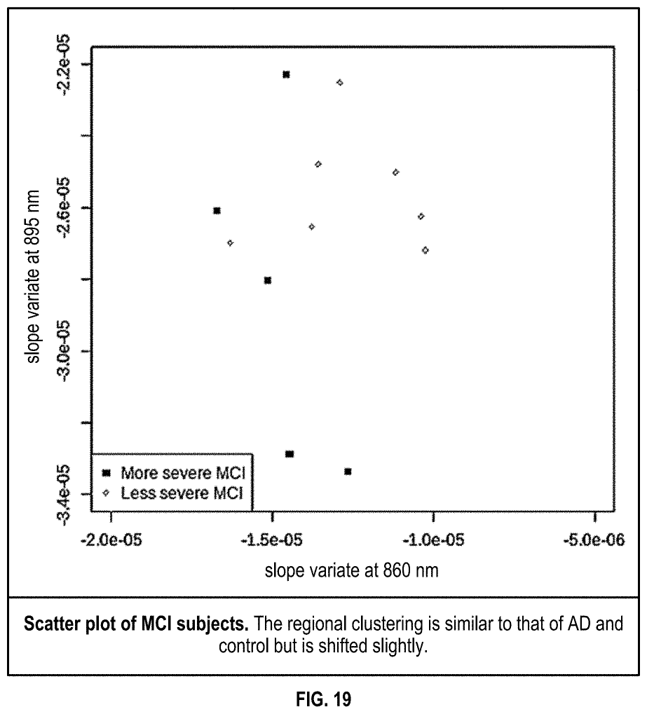

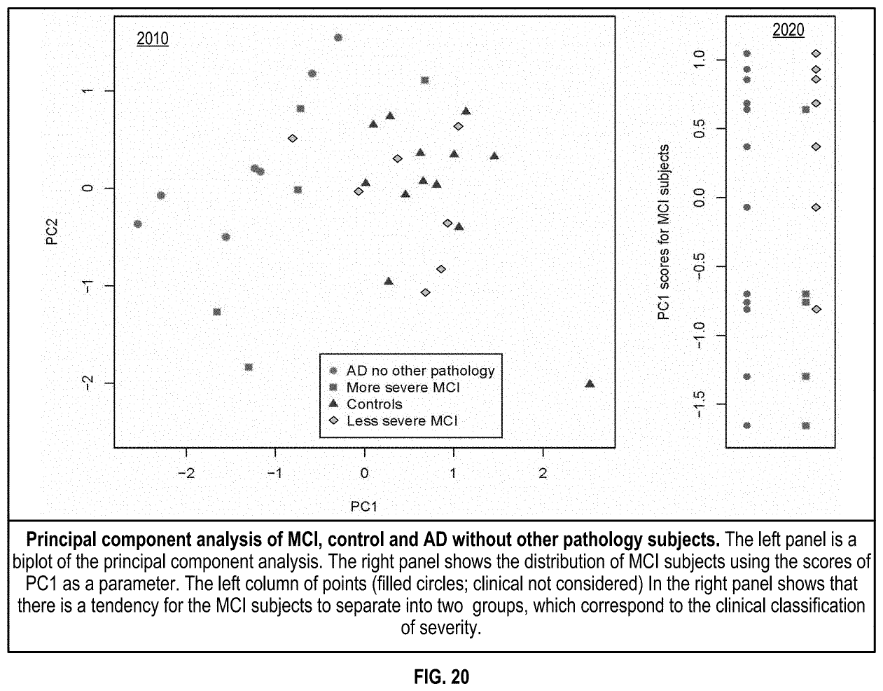

Twelve subjects with mild cognitive impairment (MCI) were also studied and clinically subclassified as more and less severe. FIG. 19. shows a plot of the results. The more and less severe MCI subjects appear to cluster similar to the AD and control scatter plots but are shifted slightly down and to the left. It is noted that the respective spans of axes (ordinate and abscissa) in each of FIGS. 17-19 are identical. Application of linear discriminant analysis to MCI and control groups suggested that 895 nm was the better discriminant (ratio of weights: 1.3), whereas MCI and pure AD returned 860 nm as the better (ratio of weights: 8.2). To determine whether proper weighting of the two slope variates might clarify the relationship among MCI, control, and pure AD, principal component analysis (PCA) can be performed on those three groups. A computing device, e.g, the computing device 201 (FIG. 2), can perform the PCA. More specifically, the fraction-product determination software 206 (FIG. 2), in response to being executed by one or more processors of the computing device, can perform the PCA. PCA is a method of data analysis that in this setting may be viewed as fitting a straight line to the data by the method of least squares. This first straight line and a second straight line normal to the first straight line become a new set of axes for displaying the data in FIG. 20. The new axes are referred to as first principal component (PC1) and second principal component (PC2). The new co-ordinates of the points are often called “scores.” The new axes are mean-centered (the mean being for all three groups) and are a linear combination of the previous axes, that is, two different weighted combinations of the two slope variates (FIG. 20, panel 2010). For the data, what is most important is that the classification of a point (as one of AD, MCI, or control) plays no role in determining either the principal components or the scores.

Examining the MCI scores presented on panel 2020 in FIG. 20 simply as points (filled circles) leads to the conclusion that they tend to cluster into two groups; by the contrary hypothesis, they would have clustered about their mean value in the new coordinates. When clinical assessments are factored in, it is clear that the clusters correspond to those that are more and less impaired, with two exceptional points (FIG. 20, panel 2020). In other words, the most positive represent the “more severe” (filled square) and the most negative represent “less severe” (open diamond) in the right hand column of the right panel in FIG. 20. When the PCI scores are used to characterize the MCI subjects that have been clinically sub-classified, the separation of the more and the less impaired is statistically significant (Welch's t-test, p<0.041), even though two subjects are misclassified.

### B. Discussion

Starting with 5 control subjects and 5 subjects with pure AD, two spectral features that distinguish AD from control are determined. Excluding those AD subjects with other significant pathology from the search aided in identifying the features because the additional pathology caused many confounding optical signals. All of the 13 subjects subsequently plotted in FIG. 18 played no role in the determination of the features, but roughly followed the same clustering in the diagram. This marks the first successful classification of a neurodegenerative condition in vivo by near-infrared spectroscopy. FIG. 18 does demonstrate that, when viewed from the proper mathematical perspective based on the fraction-product, Alzheimer's pathology can be observed through the near-infrared window.

The fact that PCA shows two clusters of the MCI values that correspond to AD and control groups is important in potential applications. In a physical sense, it means that some MCI patients have brains that are similar to AD brains whereas others have brains similar to controls. These mathematical results are independent of clinical assessments but are consistent with the clinical assessments. Although FIG. 19 suggests this finding, PCA makes it quantitative. It is noted that the slope variate at 895 nm plays a more important role in distinguishing MCI from control subjects, whereas the slope variate at 860 nm better separates MCI from those with AD. This suggests that the approach of this disclosure probes two distinct processes with that at 895 nm being more significant earlier in the progression of AD and that at 860 nm playing a more important role later in the progression of AD. No biochemical structure has been identified that could underlie the signals at 895 nm and 860 nm. Without speculation, it opens the possibility of discovering a new factor in the pathology of Alzheimer's disease.

More certain is it that larger clinical studies on MCI patients can determine whether the optical spectral changes track the progression of the disease in a useful manner. The methods, apparatuses, computer-readable media, and systems described herein provide a safe, non-invasive technique for assessing response to treatments in real-time, as the treatment is implemented. An example application scenario is where the signal at 895 nm responds to an intervention that prevents the progression of the 860 nm signal. In that scenario, an Alzheimer's survivor may be identified.

### C. Methods

### Human Subjects

Subjects were recruited as part of an ongoing project to monitor the progression of neurodegenerative diseases, especially Alzheimer's disease. All subjects were recruited through a process approved by the Institutional Review Board; informed consent was obtained in all cases. Subjects clinically diagnosed with senile dementia of the Alzheimer's type (SDAT) were recruited from the inpatient dementia unit of the Edith Nourse Rogers Memorial Veterans Hospital. All autopsies were performed by the same neuropathologist. Control subjects and those with mild cognitive impairment had volunteered for the Health Outreach Program for the Elderly (HOPE) of the Boston University Alzheimer's Disease Center. Participants in the HOPE protocol are to undergo cognitive assessment yearly. The results are reviewed and an expert review panel assigns a consensus diagnosis. The control subjects were younger (mean age 76.4 years) than the Alzheimer's subjects (mean age 82.3 years) but it is not believed that this is clinically significant for these data

Exclusion criteria. 25 dementia subjects came to autopsy. Five were excluded from the analysis described herein. Two had Lewy bodies present in the temporal isocortex, which is included in the light field interrogated by the spectroscopic technique disclosed herein. This adds a confounding factor to the signal. The subjects with Lewy bodies in FIG. 18 had none present in the temporal isocortex. Three excluded subjects had frontotemporal lobar degeneration with highly varied additional pathology. These groups had too few members and too much heterogeneity to be included here. None of the control or mildly cognitively impaired subjects was excluded.

### Spectroscopy

Measurements can be made in two sessions, approximately two years apart, for example. The room where a subject being probed is located can be darkened to minimize ambient light. In some embodiments, optical measurements can be made through fiber optic cables made of silica with a low hydroxyl concentration, with a diameter of 600 μm and with an NA of 0.22. The disclosure is not limited to optics having such attributes. Other optics can be utilized to collect spectroscopic data in vivo in accordance with aspects of this disclosure. A plastic template probe (e.g., template probe 1400 (FIG. 14)) can be constructed by drilling a first hole for the illuminating fiber (source optical fiber) and five holes at distances of 10 mm, 15 mm, 20 mm, 25 mm, and 30 mm from the first hole for the various positions of the readout optical fiber. This disclosure is not limited in that respect and other configurations of plastic template probes can be constructed and used in spectroscopic measurements. The plastic template probe can be secured to the subject's temple by an elastic band around the subject's head. The disclosure is not limited to such template structure (or template probe) and/or spectroscopy equipment.

The readout fiber conducted the reflected light to an imaging spectrograph (Kaiser Optical Systems, Ann Arbor, Mich., USA) that uses a camera cooled to −50° C. (Andor Technologies, South Windsor, Conn., USA) as light detector device. A 20 W tungsten lamp (Ocean Optics, Dunedin, Fla., USA) served as light source in the optical measurements. At each source-readout (or source-detector) separation, two optical spectra were collected, one at each of the subject's temple. The 25 mm source-detector separation optical spectra were analyzed. The disclosure, however, is not limited to analysis for such source-detector separation. Each spectrum can be corrected for acquisition time and background; correction for lamp output and detector response was achieved by a reference spectrum obtained by reflection from barium sulfate (first session) or a Spectralon low reflectance standard (second session). To calculate the first derivative of the spectral intensity at a particular pixel, the optical spectrum can be smoothed by boxcar averaging and a slope at the particular pixel was determined by performing a least-squares fit of a straight line through a region of 11 pixels about the particular pixel and then assigning the slope of the straight line to the slope at the particular pixel. That number of pixels is simply illustrative and more or fewer that 11 pixels can be considered. A computing device, e.g, the computing device 201 (FIG. 2), can determine such a first derivative. More specifically, the fraction-product determination software 206 (FIG. 2), in response to being executed by one or more processors of the computing device, can determine such a derivative. For all subjects, slope variates from both temples are averaged before analysis. Seven subjects were measured in both series and both values for each temple were averaged before analysis. That number of subject is simply illustrative and more or fewer that seven subjects can be measured.

### Feature Selection

Although the general principle is to select those features that maximally separate the groups of interest, various approaches can be utilized to achieve this. An approach is to maximize the Mahalanobis distance among the groups of interest, and for the univariate case with two groups, this reduces to the t-statistic calculated on the two means and variances. At each pixel, the t-statistic was determined for the pure AD group and control group; those features (or pixels) with the highest t-values became candidates to be used for classification. However, many high t-values were due to outliers and were rejected as useful features. To solve this problem, the methods, apparatuses, computer-readable media, and systems described herein implement a median-based approach in which the two group medians were determined and an estimated cut-off determined by the average of the two group medians. The fraction of each group correctly classified was determined and their product was taken as the measure of separation between the two groups. The median-based approach provides the same results as the t-statistics but with much more efficiency. Because optical phenomena have linewidth, features that show significant efficacy over several contiguous pixels were used/desired. The methods, apparatuses, computer-readable media, and systems described herein suggest regions around 895 nm and 860 nm as giving the best separation of AD from control. A computing device, e.g, the computing device 201 (FIG. 2) can implement such a median-based approach. More specifically, the fraction-product determination software 206 (FIG. 2), in response to being executed by one or more processors of the computing device, can implement such a median-based approach.

### Statistical Analysis

As is described herein, the fraction-product determination software 206 (FIG. 2) can include multiple components that, in response to being executed by one or more processors, can perform various analyses (including statistical analyses) described herein. At least one first component of the multiple components can perform all statistical calculations. For example, the at least one first component can embody R statistical software (R: A Language and Environment for Statistical Computing). At least one second component of the multiple components can perform linear discriminant analysis (LDA) and principal component analysis (PCA). For example, the at least one second component can embody components in the “MASS” library for linear discriminant analysis (LDA) and principal component analysis (prcomp with scale=T). It is noted that LDA was not used to calculate scores but only to determine the relative weight of each variate in distinguishing two groups. In some cases, the at least one first component can perform Hotelling T2 tests. For example, the at least one first component can include the “Hotelling” package for Hotelling T2 test.

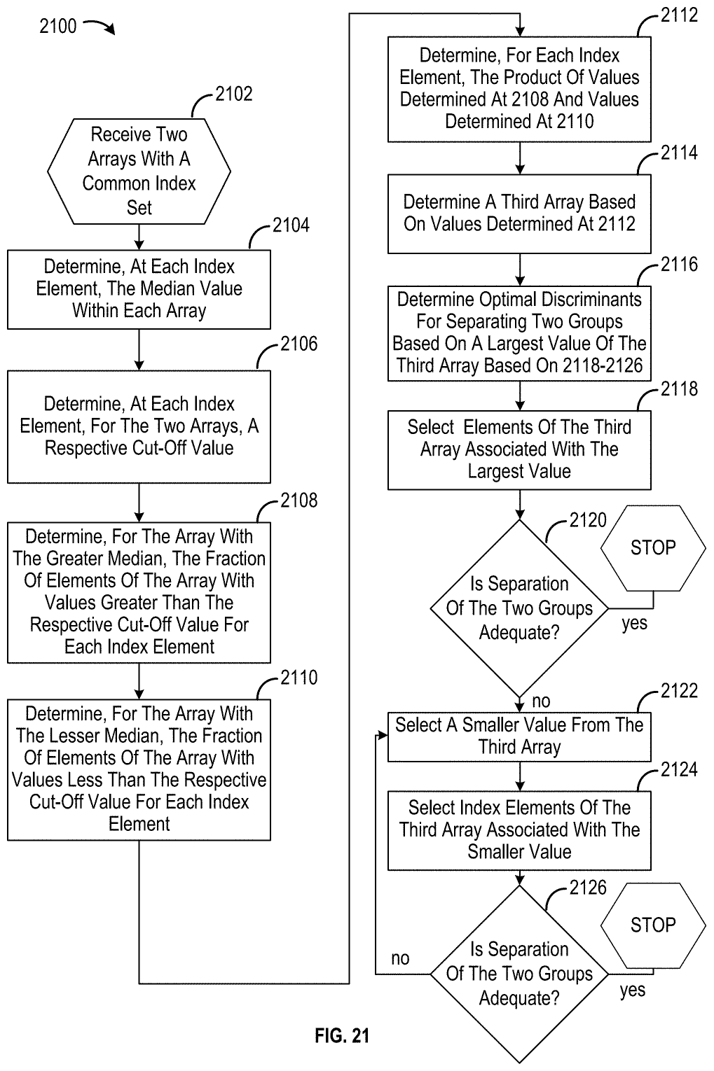

In view of the aspects described herein, example methods that may be implemented in accordance with this disclosure can be better appreciated with reference, for example, to the flowcharts in FIGS. 21-23. For the sake of simplicity of explanation, the example methods disclosed herein are presented and described as a series of blocks (with each block representing one or more actions or operations in a method, for example). However, the example methods are not limited by the order of blocks and associated actions or operations, as some blocks may occur in different orders and/or concurrently with other blocks from those that are shown and described herein. Further, not all illustrated blocks, and associated action(s), may be required to implement an example method in accordance with one or more aspects of the disclosure. Two or more of the example methods (and any other methods disclosed herein) may be implemented in combination with each other. It is noted that the example methods (and any other methods disclosed herein) may be alternatively represented as a series of interrelated states or events, such as in a state diagram.

FIG. 21 shows a flowchart of an example method 2100 for evaluating brain tissue and material based on a fraction-product. The example method 2100 may be used to determine optimal discriminants for two groups (for separating the two groups, for example) given numerical measurements of particular properties of members of both groups. Separable groups may include specimens with Alzheimer's disease vs. normal (age-matched) specimens, specimens with Lewy bodies vs. specimens without Lewy bodies, specimens affected by Gulf War Illness (GWI) vs. specimens unaffected by GWI, and/or any other types of groups. A computing device or a system of computing devices can perform the example method 2100 in its entirety or partially. To that end, each one of such computing devices includes computing resources that may implement at least one of the blocks included in the example method 2100. The computing resources comprise, for example, central processing units (CPUs), graphics processing units (GPUs), tensor processing units (TPUs), memory, disk space, incoming bandwidth, and/or outgoing bandwidth, interface(s) (such as I/O interfaces or APIs, or both); controller devices(s); power supplies; a combination of the foregoing; and/or similar resources. In one example, the system of computing devices may include programming interface(s); an operating system; software for configuration and/or control of a virtualized environment; firmware; and similar resources.

In some embodiments, the computing device that implements the example method 2100 may be embodied in, or can constitute, the computing device 201 (FIG. 2). The computing device can thus host the fraction-product determination software 206 (FIG. 2) and can implement the example method 2100 by executing one or more instances of the fraction-product determination software 206 (FIG. 2). Thus, in response to execution, the fraction-product determination software 206 (FIG. 2) can perform the operations corresponding to the blocks, individually or in combination, of the example method 2100.

At block 2102, the computing device (e.g., a data analysis device, a smart device, a device configured with artificial intelligence, etc.) can receive two arrays/matrices of numerical data. For example, the computing device can execute, or can continue executing, the fraction-product determination software 206 (FIG. 2) to receive such optical spectra. The numerical data may be based on one or more properties measured and/or determined using a spectrograph and/or the like. The two arrays/matrices. may be associated with a common index set. For example, the common index set may include a name or characterization of the discriminant property.

At block 2104, at each index element, the computing device can determine (via the fraction-product determination software 206 (FIG. 2), for example) the median value within each of the two arrays. Index elements may represent and/or may be based on, for example, wavelengths within the NIR window over which near-infrared reflectance spectroscopy is applied to the human temple. The index elements may be determined using feature selection on a first derivative of a normalized intensity spectrum obtained from the near-infrared reflectance spectroscopy. Measured properties may be and/or may represent the determined spectral intensities, and/or the like.

At block 2106, the computing device can determine (via the fraction-product determination software 206 (FIG. 2), for example) the average value of the two medians for each index element. That average value of the two medians may be designated a cut-off value for that index element.

At block 2108, for the array with the greater median, the computing device can determine (via the fraction-product determination software 206 (FIG. 2), for example) the fraction of the elements of the array with values greater than the cut-off value. The fraction of the elements of the array with values greater than the cut-off value may be determined and designated f_{g}.

At block 2110, for the array with the lesser median, the computing device can determine (via the fraction-product determination software 206 (FIG. 2), for example) the fraction of the elements of that array with values less than the cut-off value. The fraction of the elements of the array with values less than the cut-off value may be determined and designated f_{g}.

At block 2112, for each index element, the computing device can determine (via the fraction-product determination software 206 (FIG. 2), for example) the product of f_{g} and f ₗ and can designate that product as the fraction-product. It is noted that when evaluating optical spectra, there may be several wavelengths with fraction-product values above the cut-off value(s). For example, in a scenario where there are three wavelengths with fraction-product values above the cut-off value(s), a three-dimensional (3D) plot of the wavelengths may be reduced to a two-dimensional (2D) plot. Reducing the 3D plot to a 2D plot may be performed by principal component analysis (PCA).

At block 2114, the computing device can determine or otherwise generate (via the fraction-product determination software 206 (FIG. 2), for example) a third array with the common index set based on the fraction-product values.

At block 2116, the computing device can determine (via the fraction-product determination software 206 (FIG. 2), for example) optimal discriminants (or particular index elements) for separating the two groups. In some cases, the optimal discriminants may be determined by selecting the largest value of the fraction-product in the third array, based on 2118-2126.

At block 2118, the computing device can select (via the fraction-product determination software 206 (FIG. 2), for example) index elements associated with the largest value of the fraction-product in the third array.

At block 2120, the computing device can assess (via the fraction-product determination software 206 (FIG. 2), for example) the separation of the two groups based on the index elements associated with the largest value of the fraction-product in the third array. The assessment can be based, in some cases, on analysis described in connection with FIG. 9 and FIG. 11. In some cases, the computing device can determine that the separation of the two groups is adequate. In some cases, the separation can be adequate when it justifies a performing a clinical trial. The example method 2100 may then stop and the two groups may be separated.

In other cases, the computing device can determine that the separation of the two groups is not adequate. Responsive to that negative determination, the computing device can select (via the fraction-product determination software 206 (FIG. 2), for example) a smaller value of the fraction-product at block 2122.

At block 2124, the computing device can select (via the fraction-product determination software 206 (FIG. 2), for example) index elements associated with the smaller value of the fraction-product in the third array.

At block 2126, the computing device can assess (via the fraction-product determination software 206 (FIG. 2), for example) the separation of the two groups based on the index elements associated with the smaller value of the fraction-product in the third array. In cases where it is determined that the separation is adequate, the example method 2100 may stop and the two groups may be separated. In other case where the separation is not adequate, flow of the example method 2100 can return to block 2122 and the example method 2100 may be repeated from block 2122.

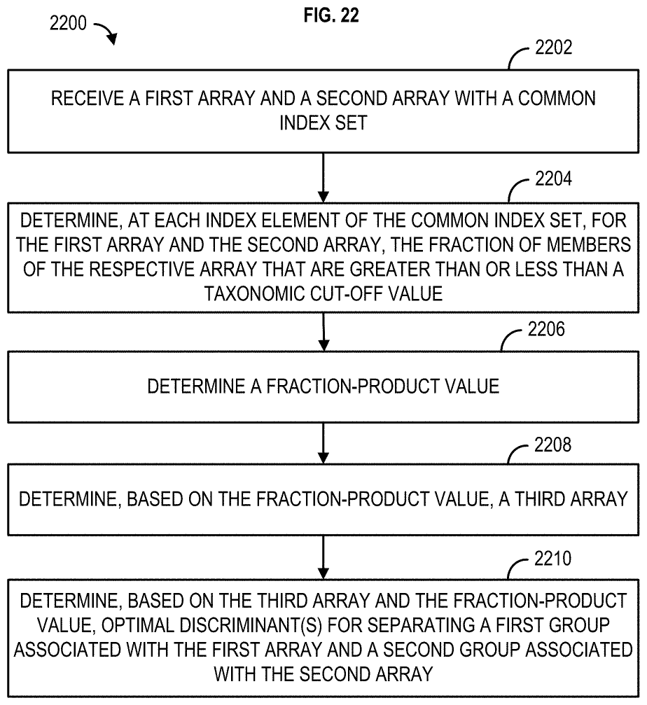

FIG. 22 shows a flowchart of an example method 2200 for evaluating brain tissue and material based on a fraction-product. The example method 2200 may be used to determine optimal discriminants for two groups (for separating the two groups, for example) given numerical measurements of particular properties of members of both groups. Separable groups may include specimens with Alzheimer's disease vs. normal (age-matched) specimens, specimens with Lewy bodies vs. specimens without Lewy bodies, specimens affected by Gulf War Illness (GWI) vs. specimens unaffected by GWI, and/or any other types of groups. A computing device or system of computing devices can perform the example method 2200 in its entirety or partially. To that end, each one of such computing devices includes computing resources that may implement at least one of the blocks included in the example method 2200. The computing resources comprise, for example, CPUs, GPUs, TPUs, memory, disk space, incoming bandwidth, and/or outgoing bandwidth, interface(s) (such as I/O interfaces or APIs, or both); controller devices(s); power supplies; a combination of the foregoing; and/or similar resources. In one example, the system of computing devices may include programming interface(s); an operating system; software for configuration and/or control of a virtualized environment; firmware; and similar resources.

In some embodiments, the computing device may be embodied in, or can constitute, the computing device 201 (FIG. 2). The computing device can thus host the fraction-product determination software 206 (FIG. 2) and can implement the example method 2200 by executing one or more instances of the fraction-product determination software 206 (FIG. 2). Thus, in response to execution, the fraction-product determination software 206 (FIG. 2) can perform the operations corresponding to the blocks, individually or in combination, of the example method 2200. In some cases, the computing device that performs the example method 2200 can be the same computing device that performs the example method 2100 (FIG. 21).

At block 2202, the computing device (e.g., a data analysis device, a smart device, a device configured with artificial intelligence, etc.) can receive a first array of numbers and a second array of numbers. For example, the computing device can execute, or can continue executing, the fraction-product determination software 206 (FIG. 2) to receive such optical spectra. The first array and the second array are associated with a common index set, where the common index set contains a plurality of index elements. The first array of numbers and the second array of numbers may be associated with optical spectra.

At block 2204, the computing device can determine (via the fraction-product determination software 206 (FIG. 2), for example): (a) at each index element of the plurality of index elements, for the first array and the second array, a median of numeric values of members of the respective array, (b) at each index element of the plurality of index elements, based on an average of the two median values determined at (a), a taxonomic cut-off value, (c) for the respective array associated with the greater median value between the two median values, a fraction of the members of the respective array having numeric values that exceed the taxonomic cut-off value determined at (b), and (d) for the respective array associated with the lesser median value between the two median values, the fraction of the members of the respective array having numeric values that are less than the taxonomic cut-off value determined at (b).

At block 2206, the computing device can determine (via the fraction-product determination software 206 (FIG. 2), for example), at each index element of the plurality of index elements, a respective fraction-product value. The respective fraction-product value is based on a product of the respective value determined at block 2204(c) and the respective value determined at block 2204(d).

At block 2208, the computing device can determine (via the fraction-product determination software 206 (FIG. 2), for example), based on each of the respective fraction-product values, a third array of numbers associated with the common index set.

At block 2210, the computing device can determine (via the fraction-product determination software 206 (FIG. 2), for example), based on the third array, one or more optimal discriminants for separating a first group and a second group. The first group is associated with the first array and the second group is associated with the second array. The one or more optimal discriminants are equal to a largest value of the third array.

Determining the one or more optimal discriminants may include: (1) selecting the largest fraction-product value of the fraction-product values associated with the third array; (2) selecting index elements of the third array associated with the largest fraction-product value; (3) assessing, based on the index elements of the third array selected at step (2), a separation of the first group and the second group; (4) if the separation is inadequate or otherwise unsatisfactory, determining a fraction-product value less than the largest fraction-product value; (5) selecting index elements of the third array associated with the fraction-product value less than the largest fraction-product value; (6) assessing, based on the index elements of the third array selected at step (5), a separation of the first group and the second group; and (7) if the separation is inadequate, repeating steps (4)-(7).

The example method 2200 may further include determining that the fraction-product value exceeds a value for one or more contiguous index elements of the common index set, and selecting, based on the fraction-product value exceeding the value for the one or more contiguous index elements of the common index set, a feature of optical spectra.

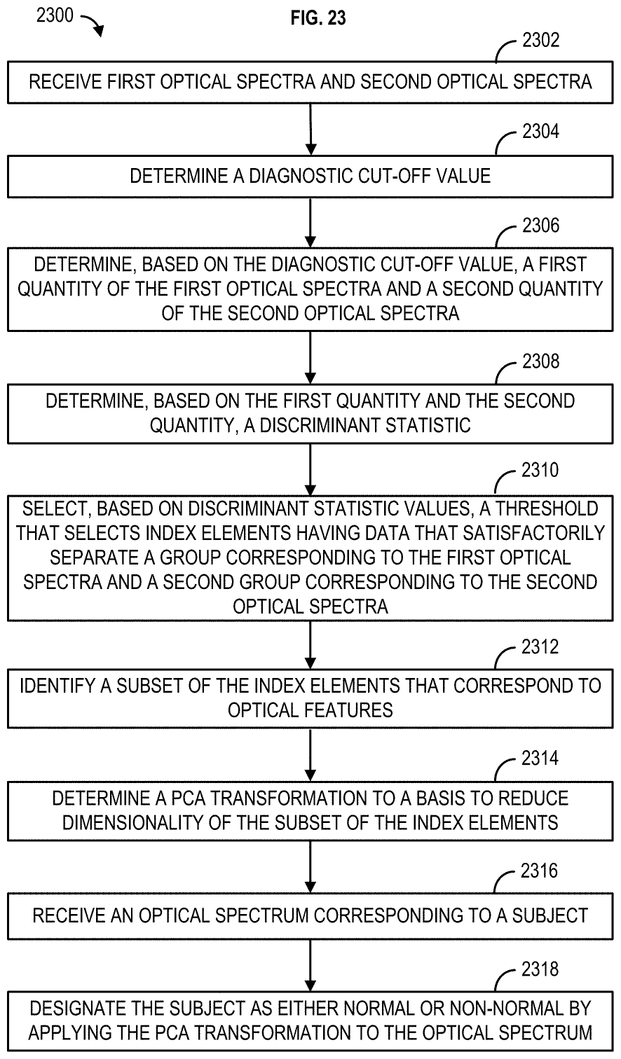

FIG. 23 shows a flowchart of an example method 2300 for evaluating brain tissue and material based on a fraction-product and optical spectroscopy. The example method 2300 may be used to determine optimal discriminants for two groups (for separating the two groups) given numerical measurements of particular properties of members of both groups. Separable groups may include specimens with Alzheimer's disease vs. normal (age-matched) specimens, specimens with Lewy bodies vs. specimens without Lewy bodies, specimens affected by Gulf War Illness (GWI) vs. specimens unaffected by GWI, and/or any other types of groups. A computing device or system of computing devices can perform the example method 2200 in its entirety or partially. To that end, each one of such computing devices includes computing resources that may implement at least one of the blocks included in the example method 2300. The computing resources comprise, for example, CPUs, GPUs, TPUs, memory, disk space, incoming bandwidth, and/or outgoing bandwidth, interface(s) (such as I/O interfaces or APIs, or both); controller devices(s); power supplies; a combination of the foregoing; and/or similar resources. In one example, the system of computing devices may include programming interface(s); an operating system; software for configuration and/or control of a virtualized environment; firmware; and similar resources.

In some embodiments, the computing device may be embodied in, or can constitute, the computing device 201 (FIG. 2). The computing device can thus host the fraction-product determination software 206 (FIG. 2) and can implement the example method 2300 by executing one or more instances of the fraction-product determination software 206 (FIG. 2). Thus, in response to execution, the fraction-product determination software 206 (FIG. 2) can perform the operations corresponding to the blocks, individually or in combination, of the example method 2300. In some cases, the computing device that performs the example method 2300 can be the same computing device that performs the example method 2100 (FIG. 21) or the example method 2200 (FIG. 22), or both.

At block 2302, the computing device (e.g., a data analysis device, a smart device, a device configured with artificial intelligence, etc.) can receive first optical spectra and second optical spectra. For example, the computing device can execute, or can continue executing, the fraction-product determination software 206 (FIG. 2) to receive such optical spectra. The first optical spectra may correspond to one or more first specimens (or, in some cases, subjects) having a medical condition, and the second optical spectra may correspond to one or more second specimens (or, in some cases, subjects) that do not have the medical condition. The medical condition may be a neuropathologic condition, and may include one or more of Alzheimer's disease, one or more Lewy bodies in brain tissue, or Gulf War Illness.

At block 2304, the computing device can determine (via the fraction-product determination software 206 (FIG. 2), for example), based on an average of median values of optical spectral intensity for wavelengths present in each of the first optical spectra and each of the second optical spectra, a diagnostic cut-off value.

At block 2306, the computing device can determine (via the fraction-product determination software 206 (FIG. 2), for example), based on the diagnostic cut-off value, a first quantity of the first optical spectra and a second quantity of the second optical spectra. The first quantity of the first optical spectra may include median values of optical spectral intensity that are less than or equal to the diagnostic cut-off value. The second quantity of the second optical spectra may include median values of optical spectral intensity that are less than or equal to the diagnostic cut-off value.

At block 2308, the computing device can determine (via the fraction-product determination software 206 (FIG. 2), for example), based on the first quantity and the second quantity, a discriminant statistic. The discriminant statistic may include a product of the first quantity and the second quantity, in some cases.

At block 2310, the computing device can select (via the fraction-product determination software 206 (FIG. 2), for example), based on discriminant statistic values, a threshold value that selects index elements having data (e.g., spectroscopic data) that satisfactorily separate (e.g., maximally separate) a group of subjects corresponding to the first optical spectra and a second group of subjects corresponding to the second optical spectra. The threshold value may be, for example, 0.45. The threshold value may be any value.

At block 2312, the computing device can identify or otherwise determine (via the fraction-product determination software 206 (FIG. 2), for example) a subset of the index elements that correspond to optical features in accordance with this disclosure. That subset can be used for medical diagnosis in conjunction with optical spectra (e.g., NIR reflectance spectra) corresponding to subjects to be diagnosed.

At block 2314, the computing device can determine (via the fraction-product determination software 206 (FIG. 2), for example) a PCA transformation to a principal-component basis to reduce dimensionality of the subset of the index elements. In some cases, block 2314 and block 2310 embody block 106 (FIG. 1). After the PCA transformation has been determined, the computing device can reduce the dimensionality of the subset of the candidate discriminants by applying the PCA transformation to the subset of the candidate discriminants.

At block 2316, the computing device can receive an optical spectrum corresponding to a subject (e.g., subject 240 (FIG. 2)) to be diagnosed as having a medical condition or not having the medical condition. The subject can be an unknown subject and can be a living. As mentioned, the medical condition may be a neuropathologic condition and may include AD, Lewy bodies, GWI, a combination thereof, or similar, for example. As is described herein, the optical spectrum can be a NIR reflectance spectrum measured at the subject's temple(s) in vivo. The optical spectrum can be obtained, at least partially, using a template probe (e.g., template probe 1400 (FIG. 14)) that injects light into the subject's brain using a first optical fiber (e.g., a source optical fiber 224 (FIG. 2)) and collects reflected light using a second optical fiber (e.g., a readout optical fiber 226 (FIG. 2)), a light detector, and a spectrometer, among other optics and sensor devices.

At block 2318, the computing device can designate (via the fraction-product determination software 206 (FIG. 2), for example) the subject as either normal or non-normal. To that end, the computing device can determine that the subject pertains to a group of normal subjects or a group of non-normal subjects by applying the PCA transformation to the optical spectrum. In some embodiments, block 2318 can constitute block 110 (FIG. 1). Normal subjects are not afflicted by the medical condition and non-normal subjects are afflicted by the medical condition.

The method 2300 may also include determining one or more wavelengths present in each of the first optical spectra and one or more wavelengths present in each of the second optical spectra, and determining, based on the one or more wavelengths present within each of the first optical spectra and the second optical spectra, the median value of optical spectral intensity for each of the first optical spectra and the second optical spectra.

Blocks 2302 to 2310 can collectively embody an example discovery process, and blocks 2312 to 2318 can collectively embody an example diagnosis process. The disclosure is not limited to diagnosis processes that use PCA and/or LDA in accordance with aspects described herein. Indeed, other diagnosis processes also can be implemented by applying machine-learning techniques to spectroscopic data and one or more candidate optical discriminants.

As described herein, finding optical features (or optical discriminants) that are useful for classification of a subject as pertaining to a normal group or a non-normal group can permit implementing a practical diagnostic technique. Further, in connection with neurological disorders, for example, such a technique can be readily implemented in vivo and, thus, can be more efficient and superior to existing diagnostic techniques. For example, in the study of Alzheimer's disease against controls, two candidate classifiers completely separated the two groups (FIG. 18). A clinical trial using the decision boundary 1810 in FIG. 18 may not show complete separation. Yet, the features generated by applying fraction-product in accordance with this disclosure can remain useful. To that end, a practical diagnostic technique can incorporate a gray zone in which subjects may not be classifiable. Similarly, in classification of subjects with/without Lewy bodies, the application of fraction-product in accordance with this disclosure yields three features (or optical discriminants). PCA reduced the dimension from 3 to 2 while retaining 79% of the variance. Panel 1610 (FIG. 16) shows that the PCA scores clearly separate the Lewy body from non-Lewy body subjects. That is, embodiments of this disclosure can generate a decision line 1615 (FIG. 16) in the two-dimensional component space. Embodiments of this disclosure can implement LDA to reduce the dimension to 1, thus further defining that separation (panel 1620 (FIG. 16)). A clinical trial for these three features has more options for evolution; it could be 1D or 2D or require a gray zone.

As is used in this specification and annexed drawings, the terms “module,” “component,” “system,” “platform,” and the like, can refer to and/or can include a computer-related entity or an entity related to an operational machine with one or more specific functionalities. Such entities can be either hardware, a combination of hardware and software, software (program code or executable program code, for example), or software in execution. In one example, a component can be a process running on a processor, a processor, an object, an executable (e.g., binary software), a thread of execution, a computer program, and/or a computing device. Simply as an illustration, a software application running on a server device can be a component and the server device also can be a component. One or more modules can reside within a process and/or thread of execution. One or more components also can reside within a process and/or thread of execution. Each one of a module and a component can be localized on one computing device and/or distributed between two or more computing devices. In another example, respective components (or modules) can execute from various computer-readable storage media having various data structures stored thereon. The components (or modules) can communicate via local and/or remote processes such as in accordance with a signal having one or more data packets (e.g., data from one component interacting with another component in a local system, distributed system, and/or across a network such as the Internet with other systems via the signal). As another illustrations, in some cases, a component can emulate an electronic component via a virtual machine, e.g., within a cloud computing system. The terms “module” and “component” (and their plural instances) may be used interchangeably where clear from context, in some cases.

As is used in this specification and annexed drawings, the term “processor” can refer to substantially any computing processing unit or computing device, including single-core processors; single-processors with software multithread execution capability; multi-core processors; multi-core processors with software multithread execution capability; multi-core processors with hardware multithread technology; parallel platforms; and parallel platforms with distributed shared memory. Additionally, a processor can refer to electronic circuitry designed in assembled to execute code instructions and/or operate on data and signaling. Such electronic circuitry can be assembled in a chipset, for example. Accordingly, in some cases, a processor can be embodied, or can include, an application specific integrated circuit (ASIC), a digital signal processor (DSP), a field programmable gate array (FPGA), a complex programmable logic device (CPLD), a discrete gate or transistor logic, discrete hardware components, or any combination thereof designed and assembled to perform the functionality described herein. Further, in some cases, processors can exploit nano-scale architectures, such as molecular and quantum-dot based transistors, switches and gates, in order to optimize space usage or enhance performance of computing devices. A processor can also be implemented as a combination of computing processing units.

Further, in this specification and annexed drawings, terms such as “storage,” “data storage,” “repository,” and substantially any other information storage component relevant to operation and functionality of a system, subsystem, module, and component are utilized to refer to “memory components,” entities embodied in a “memory,” or components including a memory. As is described herein, memory and/or memory components of this disclosure can be either volatile memory or nonvolatile memory, or can include both volatile and nonvolatile memory. Simply as an illustration, nonvolatile memory can include read only memory (ROM), programmable ROM (PROM), electrically programmable ROM (EPROM), electrically erasable ROM (EEPROM), flash memory, or nonvolatile random access memory (RAM) (e.g., ferroelectric RAM (FeRAM). Volatile memory can include RAM, which can act as external cache memory, for example. By way of illustration and not limitation, RAM is available in many forms such as synchronous RAM (SRAM), dynamic RAM (DRAM), synchronous DRAM (SDRAM), double data rate SDRAM (DDR SDRAM), enhanced SDRAM (ESDRAM), Synchlink DRAM (SLDRAM), direct Rambus RAM (DRRAM), direct Rambus dynamic RAM (DRDRAM), and Rambus dynamic RAM (RDRAM). Embodiments of this disclosure are not limited to these types of memory, and other types of memory devices can be contemplated.

While specific configurations have been described, it is not intended that the scope be limited to the particular configurations set forth, as the configurations herein are intended in all respects to be possible configurations rather than restrictive.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic concerning an arrangement of steps or operational flow; plain meaning derived from grammatical organization or punctuation; the number or type of configurations described in the specification.

It will be apparent to those skilled in the art that various modifications and variations may be made without departing from the scope or spirit. Other configurations will be apparent to those skilled in the art from consideration of the specification and practice described herein. It is intended that the specification and described configurations be considered as exemplary only, with a true scope and spirit being indicated by the following claims.

What is claimed is:

1. A computer-implemented method comprising:
receiving, by a computing device, a first array of numbers and a second array of numbers, wherein the first array and the second array are associated with a common index set, wherein the common index set is associated with a plurality of index elements;
determining:
a) at each index element of the plurality of index elements, for the first array and the second array, a median of numeric values of members of the respective array,
b) at each index element of the plurality of index elements, based on an average of the two median values determined at (a), a taxonomic cut-off value,
c) for the respective array associated with the greater median value between the two median values, a fraction of the members of the respective array whose numeric values exceed the taxonomic cut-off value determined at (b), and
d) for the respective array associated with the lesser median value between the two median values, the fraction of the members of the respective array whose numeric values are less than the taxonomic cut-off value determined at (b);
determining, at each index element of the plurality of index elements, a respective fraction-product value, wherein the respective fraction-product value is based on a product of the respective value determined at step (c) and the respective value determined at step (d);
determining, based on each of the respective fraction-product values, a third array of numbers associated with the common index set; and
determining, based on the third array, one or more optimal discriminants for separating a first group and a second group, wherein the first group is associated with the first array and the second group is associated with the second array, wherein the one or more optimal discriminants are equal to the largest value of the third array.

2. The computer-implemented method of claim 1, wherein determining the one or more optimal discriminants comprises:
1) selecting the largest fraction-product value of the fraction-product values associated with the third array,
2) selecting index elements of the third array associated with the largest fraction-product value;
3) assessing, based on the index elements of the third array selected at step (2), a separation of the first group and the second group;
4) if the separation is inadequate, determining a fraction-product value less than the largest fraction-product value;
5) selecting index elements of the third array associated with the fraction-product value less than the largest fraction-product value;
6) assessing, based on the index elements of the third array selected at step (5), a separation of the first group and the second group; and
7) if the separation is inadequate, repeating steps (4)-(7).

3. The computer-implemented method of claim 1, wherein the first array and the second array are associated with optical spectra.

4. The computer-implemented method of claim 1, further comprising:
determining that the fraction-product value exceeds a value for one or more contiguous elements of the common index set; and
selecting, based on the fraction-product value exceeding the value for the one or more contiguous elements of the common index set, a feature of optical spectra.

5. An apparatus comprising:
one or more processors; and
memory storing processor-executable instructions that, when executed by the one or more processors, cause the apparatus to:
receive, a first array and a second array, wherein the first array and the second array are associated with a common index set, wherein the common index set is associated with a plurality of index elements;
determine, at each index element of the plurality of index elements, a respective median value within the first array and a respective median value within the second array;
determine, for each index element of the plurality of index elements, a respective cut-off value, wherein the respective cut-off value comprises an average value of the respective median values within the first array and the respective median value within the second array;
determine:
a) for either the first array or the second array, based on the higher value of the respective cut-off values, a fraction of each index element of the plurality of index elements with a respective value higher than the respective cut-off value, and
b) for either the first array or the second array, based the lower value of the respective cut-off values, a fraction of each index element of the plurality of index elements with a respective value lower than the respective cut-off value,
determine, at each index element of the plurality of index elements, a respective fraction-product value, wherein the respective fraction-product value is based on a product of the respective value determined at step (a) and the respective value determined at step (b);
determine, based on each of the respective fraction-product values, a third array associated with the common index set; and
determine, based on the third array, one or more optimal discriminants for separating a first group and a second group, wherein the first group is associated with the first array and the second group is associated with the second array, wherein the one or more optimal discriminants are equal to the largest value of the third array.

6. The apparatus of claim 3, wherein the processor-executable instructions that cause the apparatus to determine the one or more optimal discriminants, further cause the apparatus to:
1) select the largest fraction-product value of the fraction-product values associated with the third array,
2) select index elements of the third array associated with the largest fraction-product value;
3) assess, based on the index elements of the third array selected at step (2), a separation of the first group and the second group;
4) if the separation is inadequate, determine a fraction-product value less than the largest fraction-product value;
5) select index elements of the third array associated with the fraction-product value less than the largest fraction-product value;
6) assess, based on the index elements of the third array selected at step (5), a separation of the first group and the second group; and
7) if the separation is inadequate, repeat steps (4)-(7).

7. One or more computer-readable media storing processor-executable instructions that, when executed by at least one processor, cause at least one processor to:
receive, a first array and a second array, wherein the first array and the second array are associated with a common index set, wherein the common index set is associated with a plurality of index elements;
determine, at each index element of the plurality of index elements, a respective median value within the first array and a respective median value within the second array;
determine, for each index element of the plurality of index elements, a respective cut-off value, wherein the respective cut-off value comprises an average value of the respective median value within the first array and the respective median value within the second array;
determine:
c) for either the first array or the second array, based on the higher value of the respective median values, a fraction of each index element of the plurality of index elements with a respective value higher than the respective cut-off value, and
d) for either the first array or the second array, based on the lower value of the respective median values, a fraction of each index element of the plurality of index elements with a respective value lower than the respective cut-off value,
determine, at each index element of the plurality of index elements, a respective fraction-product value, wherein the respective fraction-product value is based on a product of the respective value determined at step (a) and the respective value determined at step (b);
determine, based on each of the respective fraction-product values, a third array associated with the common index set; and
determine, based on the third array, one or more optimal discriminants for separating a first group and a second group, wherein the first group is associated with the first array and the second group is associated with the second array, wherein the one or more optimal discriminants are equal to a largest value of the third array.

8. The one or more computer-readable media of claim 5, wherein the processor- executable instructions that cause the at least one processor to determine the one or more optimal discriminants, further cause the at least one processor to:
1) select the largest fraction-product value of the fraction-product values associated with the third array,
2) select index elements of the third array associated with the largest fraction-product value;
3) assess, based on the index elements of the third array selected at step (2), a separation of the first group and the second group;
4) if the separation is inadequate, determine a fraction-product value less than the largest fraction-product value;
5) select index elements of the third array associated with the fraction-product value less than the largest fraction-product value;
6) assess, based on the index elements of the third array selected at step (5), a separation of the first group and the second group; and
7) if the separation is inadequate, repeat steps (4)-(7).

9. A computer-implemented method comprising:
receiving, by a computing device, first optical spectra and second optical spectra;
determining, based on an average of median values of spectral intensity for wavelengths present in each of the first optical spectra and each of the second optical spectra, a diagnostic cut-off value;
determining, based on the diagnostic cut-off value, a first quantity of the first optical spectra and a second quantity of the second optical spectra;
determining, based on the first quantity and the second quantity, a discriminant statistic; and
selecting, based on discriminant statistic values, a threshold that selects index elements having data that satisfactorily separate a group of subjects corresponding to the first optical spectra and a second group of subjects corresponding to the second optical spectra, the index elements being candidate optical discriminants.

10. The computer-implemented method of claim 9, further comprising, determining a subset of the candidate optical discriminants; and
determining a principal component analysis (PCA) transformation to a basis that reduces the dimensionality of the subject of the candidate optical discriminants; and
reducing the dimensionality of the subject of the candidate optical discriminants.

11. The computer-implemented method of claim 10, further comprising,
receiving an optical spectrum corresponding to a subject; and
designating the subject as pertaining to a group of normal subjects or a group of non-normal subjects by applying, based on the reduced subset of the candidate optical discriminants, the PCA transformation to the optical spectrum.

12. The computer-implemented method of claim 9, wherein the first optical spectra are associated with one or more first specimens having a medical condition, and wherein the second optical spectra are associated with one or more second specimens not having the medical condition.

13. The computer-implemented method of claim 12, wherein the medical condition comprises Alzheimer's disease.

14. The computer-implemented method of claim 12, wherein the medical condition comprises one or more Lewy bodies in brain tissue of the one or more first specimens.

15. The computer-implemented method of claim 12, wherein the medical condition comprises Gulf War Illness.

16. The computer-implemented method of claim 9, further comprising:
determining one or more wavelengths present in each of the first optical spectra and one or more wavelengths present in each of the second optical spectra; and
determining, based on the one or more wavelengths present within each of the first optical spectra and the second optical spectra, the median value of spectral intensity for each of the first optical spectra and the second optical spectra.

17. The computer-implemented method of claim 9, wherein the first quantity of the first optical spectra comprises median values of spectral intensity that are less than or equal to the diagnostic cut-off value.

18. The computer-implemented method of claim 9, wherein the second quantity of the second optical spectra comprises median values of spectral intensity that are less than or equal to the diagnostic cut-off value.

19. The computer-implemented method of claim 9, wherein the threshold value comprises a product of the first quantity and the second quantity.

20. The computer-implemented method of claim 19, wherein the threshold value is 0.45.

21. An apparatus comprising:
one or more processors; and
memory storing processor-executable instructions that, when executed by the one or more processors, cause the apparatus to:
receive first optical spectra and second optical spectra;
determine, based on an average of median values of spectral intensity for wavelengths present in each of the first optical spectra and each of the second optical spectra, a diagnostic cut-off value;
determine, based on the diagnostic cut-off value, a first quantity of the first optical spectra and a second quantity of the second optical spectra;
determine, based on the first quantity and the second quantity, a discriminant statistic; and
select, based on discriminant statistic values, a threshold that selects index elements having data that satisfactorily separate a group of subjects corresponding to the first optical spectra and a second group of subjects corresponding to the second optical spectra as candidate optical discriminants.

22. The apparatus of claim 21, wherein the first optical spectra are associated with one or more first specimens having a medical condition, and wherein the second optical spectra are associated with one or more second specimens not having the medical condition.

23. A computer-implemented method, comprising:
receiving an optical spectrum corresponding to a subject;
designating the subject as pertaining to a group of normal subjects or a group of non-normal subjects by applying a principal component analysis (PCA) transformation to the optical spectrum, the PCA transformation reduces a dimensionality of a set of candidate optical discriminants.

24. The computer-implemented method of claim 23, wherein the group of normal subjects comprises at least one subject not afflicted by a neurological medical condition, and wherein the group of non-normal subjects comprises at least one subject afflicted by the neurological medical condition.

25. The computer-implemented method of claim 23, further comprising,
determining the set of the candidate optical discriminants; and
determining the PCA transformation to a basis that reduces the dimensionality of the set of the candidate optical discriminants.
